SEQUENCE LISTING (115 sequences)

SEQ ID NO: 1 - DNA, 17 bp; single-stranded, linear
aarssnntngaytryga

SEQ ID NO: 2 - DNA, 17 bp; single-stranded, linear
ttrctrttrcgnggnnn

SEQ ID NO: 3 - DNA (cDNA), 131 bp; single-stranded, linear
aagggagtggactttgaggagcagcctgagcttagtctcatcctcacggctttggatggagggactccatccaggtctgggactgcattggttcaagtggaagtcatagatgccaatgacaacgcaccgta

SEQ ID NO: 4 - protein, 43 aa
KGVDFEEQPELSLILTALDGGTPSRSGTALVQVEVIDANDNAP

SEQ ID NO: 5 - DNA (cDNA), 131 bp; single-stranded, linear
aaacgcatggatttcgaggagtcttcctcctaccagatctatgtgcaagctactgaccggggaccagtacccatggcgggtcattgcaaggtgttggtggacattatagatgtgaacgacaacgcacctaa

SEQ ID NO: 6 - protein, 43 aa
KAMDFEESSSYQIYVQATDRGPVPMAGHCKVLVDIIDVNDNAP

SEQ ID NO: 7 - DNA (cDNA), 131 bp; single-stranded, linear
aagcgactggactttgagaccctgcagaccttcgagttcagcgtgggtgccacagaccatggctccccctcgctccgcagtcaggctctggtgcgcgtggtggtgctggaccacaatgacaatgcccccaa

SEQ ID NO: 8 - protein, 43 aa
KRLDFETLQTFEFSVGATDHGSPSLRSQALVRVVVLDHNDNAP

SEQ ID NO: 9 - DNA (cDNA), 131 bp; single-stranded, linear
aagggcctggattacgaggcactgcagtccttcgagttctacgtgggcgctacagatggaggctcacccgcgctcagcagccagactctggtgcggatggtggtgctggatgacaacgacaacgcccctaa

SEQ ID NO: 10 - protein, 43 aa
KGLDYEALQSFEFYVGATDGGSPALSSQTLVRMVVLDDNDNAP

SEQ ID NO: 11 - DNA (cDNA), 131 bp; single-stranded, linear
aaggcgtttgattttgaggatcagagagagttccagctaaccgctcatataaacgacggaggtaccccggttttggccaccaacatcagcgtgaacatatttgttactgaccgcaatgacaacgccccgca

SEQ ID NO: 12 - protein, 43 aa
KAFDFEDQREFQLTAHINDGGTPVLATNISVNIFVTDRNDNAP

SEQ ID NO: 13 - DNA (cDNA), 131 bp; single-stranded, linear
aaggcggtggattacgaaatcaccaagtcctatgagatagatgttcaagcccaagatctgggtcccaattctattcctgctcattgcaaaattataattaaggtcgtggatgtcaacgacaacgctcccaa

SEQ ID NO: 14 - protein, 43 aa
KAVDYEITKSYEIDVQAQDLGPNSIPAHCKIIIKVVDVNDNAP

SEQ ID NO: 15 - DNA (cDNA), 135 bp; single-stranded, linear
tatgaccatgattacgagacaaccaaagaatatacactgcggatccgggcccaggatggtggccggactccactttccaacgtctccggtctagtaaccgtgcaggtcctagacatcaacgacaatgccccccca

SEQ ID NO: 16 - protein, 44 aa
YDHDYETTKEYTLRIRAQDGGRTPLSNVSGLVTVQVLDINDNAP

SEQ ID NO: 17 - DNA (cDNA), 129 bp; single-stranded, linear
ggggggtcgattacgaggagaacggcatgttagagatcgacgtgcaggccagagacctaggacctaacccaattccagcccattgcaaggtcacagtcaagctcatcgaccgcaatgataacgccccca

SEQ ID NO: 18 - protein, 43 aa
RGVDYEENGMLEIDVQARDLGPNPIPAHCKVTVKLIDRNDNAP

SEQ ID NO: 19 - DNA (cDNA), 131 bp; single-stranded, linear
aaggggttggactacgaagacaccaaactccatgagatttacatccaggccaaagacaaaggtgccaatccggaaggagcgcattgcaaagtactggtagaggttgtggacgttaacgacaatgcccctca

SEQ ID NO: 20 - protein, 43 aa
KGLDYEDTKLHEIYIQAKDKGANPEGAHCKVLVEVVDVNDNAP

SEQ ID NO: 21 - DNA (cDNA), 131 bp; single-stranded, linear
aagggtttggactttgagcaagtagatgtctacaaaatccgcgttgacgcgacggacaaaggacaccctccgatggcaggccattgcactgttttagtgagggtattggatgaaaacgacaatgcgcctct

SEQ ID NO: 22 - protein, 43 aa
KGLDFEQVDVYKIRVDATDKGHPPMAGHCTVLVRVLDENDNAP

SEQ ID NO: 23 - DNA (cDNA), 134 bp; single-stranded, linear
aagggtatagacttcgagcagatcaaggacttcagctttcaagtggaagcccgggacgccggcagtccccaggcgctgtccggcaactgcactgtcaacatcttgatagtggatcagaacgacaacgcccctaa

SEQ ID NO: 24 - protein, 44 aa
KGIDFEQIKDFSFQVEARDAGSPQALAGNTTVNILIVDQNDNAP

SEQ ID NO: 25 - DNA (cDNA), 134 bp; single-stranded, linear
aagccgttcgactatgagcaaaccgccaacacgctggcacagattgacgccgtgctggaaaaacagggcagcaataaatcgagcattctggatgccaccattttcctggccgataaaaacgacaatgcgccaga

SEQ ID NO: 26 - protein, 44 aa
KPFDYEQTANTLAQIDAVLEKQGSNKSSILDATIFLADKNDNAP

SEQ ID NO: 27 - DNA (cDNA), 131 bp; single-stranded, linear
aagcggctggatttcgaacagttccagcagcacaagctgctcgtaagggctgttgatggaggaatgccgccactgagcagcgatgtggtcgtcactgtggatgtcaccgacctcaacgataacgcgcccta

SEQ ID NO: 28 - protein, 43 aa
KRLDFEQFQQHKLLVRAVDGGMPPLSSDVVVTVDVTDLNDNAP

SEQ ID NO: 29 - DNA (cDNA), 131 bp; single-stranded, linear
aaggggatagactttgagagtgagaattactatgaatttgatgtgcgggctcgcgatgggggttctccagccatggagcaacattgcagccttcgagtggatctgctggacgtaaatgacaacgccccact

SEQ ID NO: 30 - protein, 43 aa
KGIDFESENYYEFDVRARDGGSPAMEQHCSLRVDLLDVNDNAP

SEQ ID NO: 31 - DNA (cDNA), 131 bp; single-stranded, linear
aaggcattggactttgaggcccggcgactgtattcgctgacagttcaggccacggaccgaggcgtgccctcgctcaccgggcgtgccgaagcgcttatccagctgctagatgtcaacgacaacgcacccat

SEQ ID NO: 32 - protein, 43 aa
KALDFEARRLYSLTVQATDRGVPSLTGRAEALIQLLDVNDNAP

SEQ ID NO: 33 - DNA (cDNA), 125 bp; single-stranded, linear
aagccaattgattacgaggcaactccatactataacatggaaattgtagccacagacagcggaggtctttcgggaaaatgcactgtgtctatacaggtggtggatgtgaacgacaacgcccccaa

SEQ ID NO: 34 - protein, 41 aa
KPIDYEATPYYNMEIVATDSGGLSGKCTVSIQVVDVNDNAP

SEQ ID NO: 35 - DNA (cDNA), 446 bp; single-stranded, linear
aagcgggtagacttcgaaatgtgcaaaagattttaccttgtggtggaagctaaagacggaggcaccccagccctcagcacggcagccactgtcagcatcgacctcacagatgtgaatgataaccctcctcggttcagccaagatgtctacagtgctgtcatcagtgaggatgccttagagggggactctgtcattctgctgatagcagaagatgtggatagcaagcctaatggacagattcggttttccatcgtgggtggagatagggacaatgaatttgctgtcgatccaatcttgggacttgtgaaagttaagaagaaactggaccgggagcgggtgtcaggatactccctgctcatccaggcagtagatagtggcattcctgcaatgtcctcaacgacaactgtcaacattgatatttctgatgtgaacgacaacgcccccct

SEQ ID NO: 36 - protein, 148 aa
KRVDFEMCKRFYLVVEAKDGGTPALSTAATVSIDLTDVNDNPPRFSQDVYDAVISEDALEGDSVILLIAEDVDSKPNGQIRFSIVGGDRDNEFAVDPILGLVKVKKKLDRERVSGYSLLIQAVDSGIPAMSSTTTVNIDISDVNDNAP

SEQ ID NO: 37 - DNA (cDNA), 440 bp; single-stranded, linear
aagggggttgattatgagacaaacccacggctacgactggtgctacaggcagagagtggaggagcctttgctttctcggtgctgaccctgacccttcaagatgccaatgacaatgctccccgtttcctgcagcctcactacgtggctttcctgccagagtcccgacccttggaagggcccctgctgcaggtggaagcagacgacctggatcaaggctctggaggacagatctcctacagtctggctgcatcccagccagcacggggcttgttccatgtagacccagccacaggcactatcactaccacagccatcctggaccgggaaatctgggctgaaacacggctggtactgatggccacagacagaggaagcccagcattggtgggctcagctaccctgacagtgatggtcatcgataccaacgacaatgctcccct

SEQ ID NO: 38 - protein, 146 aa
KGVDYETNPRLRLVLQAESGGAFAFSVLTLTLQDANDNAPRFLQPHYVAFLPESRPLEGPLLQVEANDLDQGSGGQISYSLAASQPARGLFHVDPATGTITTTAILDREIWAETRLVLMATDRGSPALVGSATLTVMVIDTNDNAP

SEQ ID NO: 39 - DNA (cDNA), 124 bp; single-stranded, linear
aaggtctcgattatgaggcaactccatattataacgtggaaattgtagccacagatggtgggggcctttcaggaaaatgcactgtggctatagaagtggtggatgtgaacgacggcgctccaat

SEQ ID NO: 40 - protein, 41 aa
KGLDYEATPYYNVEIVATDGGAFDENCTVAIEVVDVNDNAP

SEQ ID NO: 41 - protein, 8 aa
DXNEXPXF

SEQ ID NO: 42 - protein, 8 aa
DXDEXPXF

SEQ ID NO: 43 - protein, 9 aa
DXNDNXPXF

SEQ ID NO: 44 - DNA (cDNA), 131 bp; single-stranded, linear
aagcggatggattttgaagacaccaaactccatgagatttacatccaggccaaagacaaaggtgccaatcccgaaggagcgcattgcaaagtacttgtagaggttgtagacgtaaacgacaacgccccagt

SEQ ID NO: 45 - protein, 43 aa
LRMDFEDTKLHEIYIQAKDKGANPEGAHCKVLVEVVDVNDNAP

SEQ ID NO: 46 - DNA (cDNA), 131 bp; single-stranded, linear
aaggctttggattacgaggatcagagagagttccaactaacagctcatataaacgacggaggtaccccagtcttagccaccaacatcagcgtgaacgtatttgttactgaccgcaatgataacgcccccta

SEQ ID NO: 47 - protein, 43 aa
KALDYEDQREFQLTAHINDGGTPVLATNISVNVFVTDRNDNAP

SEQ ID NO: 48 - DNA (cDNA), 131 bp; single-stranded, linear
aagcgcttggactacgaggagagtaacaattatgaaattcacgtggatgctacagataaaggatacccacctatggttgctcactgcaccgtactcgtgggaatcttggatgaaaatgacaacgcacccat

SEQ ID NO: 49 - protein, 43 aa
KRLDYEESNNYEIHVDATDKGYPPMVAHCTVLVGILDENDNAP

SEQ ID NO: 50 - DNA (cDNA), 131 bp; single-stranded, linear
aaaccggtggactacgagaaagtcaaagactataccatcgagatcgtggctgtggattccggcaaccctccactctctagcaccaactccctcaaggtgcaggtggtagacgtcaacgataacgcccctct

SEQ ID NO: 51 - protein, 43 aa
KPVDYEKVKDYTIEIVAVDSGNPPLSSTNSLKVQVVDVNDNAP

SEQ ID NO: 52 - DNA (cDNA), 131 bp; single-stranded, linear
aagccttttgatttcgaggacaccaaactccatgagatttacatccaggccaaagacaagggcgccaatcccgaaggagcacattgcaaagtgttggtggaggttgtggatgtgaacgacaatgcccctca

SEQ ID NO: 53 - protein, 43 aa
KPFDFEDTKLHEIYIQAKDKGANPEGAHCKVLVEVVDVNDNAP

SEQ ID NO: 54 - DNA (cDNA), 122 bp; single-stranded, linear
aaaggtgtcgattacgaggtgagtccacggctgcgactggtgctgcaggcagagagtcgaggagcctttgccttcactgtgctgaccctgaccctgcaagatgccaacgacaacgccccgag

SEQ ID NO: 55 - protein, 40 aa
KGVDYEVSPRLRLVLQAESRGAFAFTVLTLTLQDANDNAP

SEQ ID NO: 56 - DNA (cDNA), 131 bp; single-stranded, linear
aaagggattgattacgagcagttgagagacctacagctgtgggtgacagccagcgacagcggggacccgcctcttagcagcaacgtgtcactgagcctgtttgtgctggaccagaacgacaacgcccccct

SEQ ID NO: 57 - protein, 43 aa
KGIDYEQLRDLQLWVTASDSGDPPLSSNVSLSLFVLDQNDNAP

SEQ ID NO: 58 - DNA (cDNA), 125 bp; single-stranded, linear
aaggcggtcgattttgagcgcacatcctcttatcaactcatcattcaggccaccaatatggcaggaatggcttccaatgctacagtcaatattcagattgttgatgaaaacgacaacgcccccca

SEQ ID NO: 59 - protein, 41 aa
KAVDFERTSSYQLIIQATNMAGMASNATVNIQIVDENDNAP

SEQ ID NO: 60 - DNA (cDNA), 131 bp; single-stranded, linear
aaacggctagactttgaaaagatacaaaaatatgttgtatggatagaggccagagatggtggtttccctcctttctcctcttacgagaaacttgatataacagtattagatgtcaacgataacgcgcctaa

SEQ ID NO: 61 - protein, 43 aa [sequence omitted]

SEQ ID NO: 62 - DNA (cDNA), 131 bp; single-stranded, linear
aaggggatcgattatgagaaggtcaaagactacaccattgagattgtggctgtggactctggcaaccccccactctccagcactaactccctcaaggtgcaggtggtggacgtcaatgacaacgcaccgtg

SEQ ID NO: 63 - protein, 43 aa
KGIDYEKVKDYTIEIVAVDSGNPPLSSTNSLKVQVVDVNDNAP

SEQ ID NO: 64 - DNA (cDNA), 131 bp; single-stranded, linear
aagggactcgactacgaggatcggcgggaatttgaattaacagctcatatcagcgatgggggcaccccggtcctagccaccaacatcagcgtgaacatatttgtcactgatcgcaacgataatgcccccgt

SEQ ID NO: 65 - protein, 43 aa
KGLDYEDRREFELTAHISDGGTPVLATNISVNIFVTDRNDNAP

SEQ ID NO: 66 - DNA (cDNA), 470 bp; single-stranded, linear
aagggtttggactacgagaccacacaggcctaccagctcacggtcaacgccacagatcaagacaacaccaggcctctgtccaccctggccaacttggccatcatcatcacagatgtccaggacatggaccccatcttcatcaacctgccttacagcaccaacatctacgagcattctcctccgggcacgacggtgcgcatcatcaccgccatagaccaggatcaaggacgtccccggggcattggctacaccatcgtttcagggaataccaacagcatctttgccctggactacatcagcggagtgctgaccttgaatggcctgctggaccgggagaaccccctgtacagccatggcttcatcctgactgtgaagggcacggagctgaacgatgaccgcaccccatctgacgctacagtcaccacgaccttcaatatcctggttattgacatcaacgacaacgccccact

SEQ ID NO: 67 - protein, 156 aa
KGLDYETTQAYQLTVNATDQDNTRPLSTLANLAIIITDVQDMDPIFINLPYSTNIYEHSPPGTTVRIITAIDQDQGRPRGIGYTIVSGNTNSIFALDYISGVLTLNGLLDRENPLYSGGFILTVKGTELNDDRTPSDATVTTTFNILVIDINDNAP

SEQ ID NO: 68 - DNA (cDNA), 131 bp; single-stranded, linear
aagggggtcgattacgaggtactacaggcctttgagttccacgtgagcgccacagaccgaggctcaccggggctcagcagccaggctctggtgcgcgtggtggtgctggacgacaatgacaacgctcccgt

SEQ ID NO: 69 - protein, 43 aa
KGVDYEVLQAFEFHVSATDRGSPGLSSQALVRVVVLDDNDNAP

SEQ ID NO: 70 - DNA (cDNA), 131 bp; single-stranded, linear
aaggggctggattatgagcagttccagaccctacaactgggagtgaccgctagtgacagtggaaacccaccattaagaagcaatatttcactgacccttttcgtgctggaccagaatgataacgccccaaa

SEQ ID NO: 71 - protein, 43 aa
KGLDYEQFQTLQLGVTASDSGNPPLRSNISLTLFVLDQNDNAP

SEQ ID NO: 72 - DNA (cDNA), 131 bp; single-stranded, linear
aagcgggttgattacgaggatgtccagaaatactcgctgagcattaaggcccaggatgggcggcccccgctcatcaattcttcaggggtggtgtctgtgcaggtgctggatgtcaacgacaatgccccgga

SEQ ID NO: 73 - protein, 43 aa
KRVDYEDVQKYSLSIKAQDGRPPLINSSGVVSVQVLDVNDNAP

SEQ ID NO: 74 - DNA (cDNA), 125 bp; single-stranded, linear
aaaccggtagactttgagctacagcagttctatgaagtagctgtggtggcttggaactctgagggatttcatgtcaaaagggtcattaaagtgcaacttttagatgacaacgacaatgccccgat

SEQ ID NO: 75 - protein, 41 aa
KPVDFELQQFYEVAVVAWNSEGFHVKRVIKVQLLDDNDNAP

SEQ ID NO: 76 - DNA (cDNA), 125 bp; single-stranded, linear
aagggattagattttgaaactttgcccatttacacattgataatacaaggaactaacatggctggtttgtccactaatacaacggttctagttcacttgcaggatgagaatgataacgccccaaa

SEQ ID NO: 77 - protein, 41 aa
KGLDFETLPIYTLIIQGTNMAGLSTNTTVLVHLQDENDNAP

SEQ ID NO: 78 - DNA (cDNA), 134 bp; single-stranded, linear
aagcgggcggatttcgaggcgatccgggagtacagtctgaggatcaaagcgcaggacggggggcggcctcccctcagcaacaccacgggcatggtcacagtgcaggtcgtggacgtcaatgacaacgcacccct

SEQ ID NO: 79 - protein, 44 aa
KRADFEAIREYSLRIKAQDGGRPPLSNTTGMVTVQVVDVNDNAP

SEQ ID NO: 80 - DNA (cDNA), 131 bp; single-stranded, linear
aagcggttggattacgaaaaggcatcggaatatgaaatctatgttcaagccgctgacaaaggcgctgtccctatggctggccattgcaaagtgttgctggagatcgtggatgtcaacgacaacgccccctt

SEQ ID NO: 81 - protein, 43 aa
KRLDYEKASEYEIYVQAADKGAVPMAGHCKVLLEIVDVNDNAP

SEQ ID NO: 82 - DNA (cDNA), 131 bp; single-stranded, linear
aaggggatcgattatgaggatcaggtctcttacacattagcagtaacagcacatgactatggcatccctcaaaaatcagacactacctatttggaaatcttagtaattgatgttaacgacaacgcgcccca

SEQ ID NO: 83 - protein, 43 aa
KGIDYEDQVSYTLAVTAHDYGIPQKSDTTYLEILVIDVNDNAP

SEQ ID NO: 84 - DNA (cDNA), 131 bp; single-stranded, linear
aaagggttagatttcgagggcactaaagattcagcgtttaaaatagtggcagctgacacagggaagcccagcctcaaccagacagccctggtgagagtagagctggaggatgagaacgacaacgccccaat

SEQ ID NO: 85 - protein, 43 aa
KGLDFEGTKDSAFKIVAADTGKPSLNQTALVRVELEDENDNAP

SEQ ID NO: 86 - DNA (cDNA), 130 bp; single-stranded, linear
aagggtgtggattttgaaagtgtgcgtagctacaggctggttattcgtgctcaagatggaggcagcccctccagaagtaacaccacccagctcttggtcaacgtcatcgatcgaatgacaatgcgccgct

SEQ ID NO: 87 - protein, 43 aa
KGVDFESVRSYRLVIRAQDGGSPSRSNTTQLLVNVIDVNDNAP

SEQ ID NO: 88 - DNA (cDNA), 131 bp; single-stranded, linear
aagggtgtggacttcgagctgacacatctgtatgagatttggattgaggctgccgatggagacacgccaagtctgcgtagtgtaactcttataacgctcaacgtaacggatgccaatgacaatgctcccaa

SEQ ID NO: 89 - protein, 43 aa
KGVDFELTHLYEIWIEAADGDTPSLRSVTLITLNVTDANDNAP

SEQ ID NO: 90 - DNA (cDNA), 441 bp; single-stranded, linear
caaggcgtttgattttgaagagacaagtagatatgtgttgagtgtggaagctaaggatggaggagtacacacagctcactgtaatgttcaaatagaaattgttgacgagaatgacaatgccccagaggtgacattcatgtccttctctaaccagattccagaggattcagaccttggaactgtaatagccctcataaaagtgcgagacaaggattctgggcaaaatggcatggtgacatgctatactcaggaagaagttcctttcaaattagaatccacctcgaagaattattacaagctggtgattgctggagccctaaaccgggagcagacagcagactacaacgtcacaatcatagccaccgacaagggcaaaccagccctttcctccaggacaagcatcaccctgcacatctccgacatcaacgataatgcccccgt

SEQ ID NO: 91 - protein, 146 aa
KAFDFEETSRYVLSVEAKDGGVHTAHCNVQIEIVDENDNAPEVTFMSFSNQIPEDSDLGTVIALIKVRDKDSGQNGMVTCYTQEEVPFKLESTSKNYYKLVIAGALNREQTADYNVTIIATDKGKPALSSRTSITLHISDINDNAP

SEQ ID NO: 92 - DNA (cDNA), 131 bp; single-stranded, linear
aagcgagtggattacgaggccactcggaattataagctgagagttaaggctactgatcttgggattccaccgagatcttctaacatgacactgttcattcatgtccttgatgttaacgacaacgctccctt

SEQ ID NO: 93 - protein, 43 aa
KRVDYEATRNYKLRVKATDLGIPPRSSNMTLFIHVLDVNDNAP

SEQ ID NO: 94 - DNA (cDNA), 4104 bp; single-stranded, linear
  CDS 495..3572
cctctattcgacattctctttggattgttttgctataacttgaaatttgggatgtcacaaacgaaactgtcatctgtttccgccaaactgtggttctgctaatctcccaggctggcagcattggagacttgctgacttctttcatcccccactcttttcacctgaaattcctttccttggttttgctctaagtcctatgcttcagtcaggggccaaccaaatctcactgcctcctttttatcatgaagcctttgatcactgatagttctttttatatcttgaaaaatcacccttcccagtacagttaatatttagtatctctactcatcttggcacttactcacagctccataattcagtcgttttcgtacctcttcatggtgatggggagccctttggaggtggtgactgtgctttatactcctcatgatgcttcacatgtggcaggcgtggagtgcccggaggcggccctcctgattctggggcctcccaggatggagcccctgaggcacagcccaggccctggggggcaacggctactgctgccctccatgctgctagcactgctgctcctgctggctccatccccaggccacgccactcgggtagtgtacaaggtgccggaggaacagccacccaacaccctcattgggagcctcgcagccgactatggttttccagatgtggggcacctgtacaagctagaggtgggtgccccgtaccttcgcgtggatggcaagacaggtgacattttcaccaccgagacctccatcgaccgtgaggggctccgtgaatgccagaaccagctccctggtgatccctgcatcctggagtttgaggtatctatcacagacctcgtgcagaatgcgagcccccggctgctagagggccagatagaagtacaagacatcaatgacaacacacccaacttcgcctcaccagtcatcactctggccatccctgagaacaccaacatcggctcactcttccccatcccgctggcttcagaccgtgatgctggtcccaacggtgtggcatcctatgagctgcaggtggcagaggaccaggaggagaagcaaccacagctcattgtgatgggcaacctggaccgtgagcgctgggactcctatgacctcaccatcaaggtgcaggatggcggcagccccccacgcgccacgagtgccctgctgcgtgtcaccgtgcttgacaccaatgacaacgcccccaagtttgagcggccctcctatgaggccgaactatctgagaatagccccataggccactcggtcatccaggtgaaggccaatgactcagaccaaggtgccaatgcagaaatcgaatacacattccaccaggcgcccgaagttgtgaggcgtcttcttcgactggacaggaacactggacttatcactgttcagggcccggtggaccgtgaggacctaagcaccctgcgcttctcagtgcttgctaaggaccgaggcaccaaccccaagagtgcccgtgcccaggtggttgtgaccgtgaaggacatgaatgacaatgcccccaccattgagatccggggcatagggctagtgactcatcaagatgggatggctaacatctcagaggatgtggcagaggagacagctgtggccctggtgcaggtgtctgaccgagatgagggagagaatgcagctgtcacctgtgtggtggcaggtgatgtgcccttccagctgcgccaggccagtgagacaggcagtgacagcaagaagaagtatttcctgcagactaccaccccgctagactacgagaaggtcaaagactacaccattgagattgtggctgtggactctggcaaccccccactctccagcactaactccctcaaggtgcaggtggtggacgtcaatgacaacgcacctgtcttcactcagagtgtcactgaggtcgccttcccggaaaacaacaagcctggtgaagtgattgctgagatcactgccagtgatgctgactctggctctaatgctgagctggtttactctctggagcctgagccggctgctaagggcctcttcaccatctcacccgagactggagagatccaggtgaagacatctctggatcgggaacagcgggagagctatgagttgaaggtggtggcagctgaccggggcagtcctagcctccagggcacagccactgtccttgtcaatgtgctggactgcaatgacaatgaccccaaatttatgctgagtggctacaacttctcagtgatggagaacatgccagcactgagtccagtgggcatggtgactgtcattgatggagacaagggggagaatgcccaggtgcagctctcagtggagcaggacaacggtgactttgttatccagaatggcacaggcaccatcctatccagcctgagctttgatcgagagcaacaaagcacctacaccttccagctgaaggcagtggatggtggcgtcccacctcgctcagcttacgttggtgtcaccatcaatgtgctggacgagaatgacaacgcaccctatatcactgccccttctaacacctctcacaagctgctgaccccccagacacgtcttggtgagacggtcagccaggtggcagccgaggactttgactctggtgtcaatgccgagctgatctacagcattgcaggtggcaacccttatggactcttccagattgggtcacattcaggtgccatcaccctggagaaggagattgagcggcgccaccatgggctacaccgcctggtggtgaaggtcagtgaccgcggcaagcccccacgctatggcacagccttggtccatctttatgtcaatgagactctggccaaccgcacgctgctggagaccctcctgggccacagcctggacacgccgctggatattgacattgctggggatccagaatatgagcgctccaagcagcgtggcaacattctctttggtgtggtggctggtgtggtggccgtggccttgctcatcgccctggcggttcttgtgcgctactgcagacagcgggaggccaaaagtggttaccaggctggtaagaaggagaccaaggacctgtatgcccccaagcccagtggcaaggcctccaagggaaacaaaagcaaaggcaagaagagcaagtccccaaagcccgtgaagccagtggaggacgaggatgaggccgggctgcagaagtccctcaagttcaacctgatgagcgatgcccctggggacagtccccgcatccacctgcccctcaactacccaccaggcagccctgacctgggccgccactatcgctctaactccccactgccttccatccagctgcagccccagtcaccctcagcctccaagaagcaccaggtggtacaggacctgccacctgcaaacacattcgtgggcaccggggacaccacgtccacgggctctgagcagtactccgactacagctaccgcaccaacccccccaaataccccagcaagcaggtaggccagccctttcagctcagcacaccccagcccctaccccacccctaccacggagccatctggaccgaggtgtgggagtgatggagcaggtttactgtgcctgcccgtgttgggggccagcctgagccagcagtgggaggtggggccttagtgcctcaccgggcacacggattaggctgagtgaagattaagggagggtgtgctctgtggtctcctccctgccctctccccactggggagagacctgtgatttgccaagtccctggaccctggaccagctactgggccttatgggttgggggtggtaggcaggtgagcgtaagtggggagggaaatgggtaagaagtctactccaaacctaggtctctatgtcagaccagacctaggtgcttctctaggagggaaacagggagacctggggtcctgtggataactgagtggggagtctgccaggggagggcaccttcccattgtgccttctgtgtgtattgtgcattaacctcttcctcaccactaggcttctggggctgggtcccacatgcccttgaccctgacaataaagttctctatttttggaaaaaaaaaaaaaaaaaaaaaaaaaaaaaaaaaa

SEQ ID NO: 95 - protein, 1026 aa [sequence omitted]

SEQ ID NO: 96 - DNA (cDNA), 4705 bp; single-stranded, linear
  CDS 115..2827
cgaaagccatgtcggactcgtcgcccagcgcccaagcgctaacccgctgaaagtttctcagcgaaatctcagggacgatctggaccccgctgagaggaactgcttttgagtgagatggtcccagaggcctggaggagcggactggtaagcaccgggagggtagtgggagttttgcttctgcttggtgccttgaacaaggcttccacggtcattcactatgagatcccggaggaaagagagaagggtttcgctgtgggcaacgtggtcgcgaaccttggtttggatctcggtagcctctcagcccgcaggttcccggtggtgtctggagctagccgaagattctttgaggtgaaccgggagaccggagagatgtttgtgaacgaccgtctggatcgagaggagctgtgtgggacactgccctcttgcactgtaactctggagttggtagtggagaacccgctggagctgttcagcgtggaagtggtgatccaggacatcaacgacaacaatcctgctttccctacccaggaaatgaaattggagattagcgaggccgtggctccggggacgcgctttccgctcgagagcgcgcacgatcccgatctgggaagcaactctttacaaacctatgagctgagccgaaatgaatactttgcgcttcgcgtgcagacgcgggaggacagcaccaagtacgcggagctggtgttggagcgcgccctggaccgagaacgggagcctagtctccagttagtgctgacggcgttggacggagggaccccagctctctccgccagcctgcctattcacatcaaggtgctggacgcgaatgacaatgcgcctgtcttcaaccagtccttgtaccgggcgcgcgttcctggaggatgcacctccggcacgcgcgtggtacaagtccttgcaacggatctggatgaaggccccaacggtgaaattatttactccttcggcagccacaaccgcgccggcgtgcggcaactattcgccttagaccttgtaaccgggatgctgacaatcaagggtcggctggacttcgaggacaccaaactccatgagatttacatccaggccaaagacaagggcgccaatcccgaaggagcacattgcaaagtgttggtggaggttgtggatgtgaatgacaacgccccggagatcacagtcacctccgtgtacagcccagtacccgaggatgcctctgggactgtcatcgctttgctcagtgtgactgacctggatgctggcgagaacgggctggtgacctgcgaagttccaccgggtctccctttcagccttacttcttccctcaagaattacttcactttgaaaaccagtgcagacctggatcgggagactgtgccagaatacaacctcagcatcaccgcccgagacgccggaaccccttccctctcagcccttacaatagtgcgtgttcaagtgtccgacatcaatgacaaccctccacaatcttctcaatcttcctacgacgtttacattgaagaaaacaacctccccggggctccaatactaaacctaagtgtctgggaccccgacgccccgcagaatgctcggctttctttctttctcttggagcaaggagctgaaaccgggctagtgggtcgctatttcacaataaatcgtgacaatggcatagtgtcatccttagtgcccctagactatgaggatcggcgggaatttgaattaacagctcatatcagcgatgggggcaccccggtcctagccaccaacatcagcgtgaacatatttgtcactgatcgcaatgacaatgccccccaggtcctatatcctcggccaggtgggagctcggtggagatgctgcctcgaggtacctcagctggccacctagtgtcacgggtggtaggctgggacgcggatgcagggcacaatgcctggctctcctacagtctctttggatcccctaaccagagcctttttgccatagggctgcacactggtcaaatcagtactgcccgtccagtccaagacacagattcacccaggcagactctcactgtcttgatcaaagacaatggggagccttcgctctccaccactgctaccctcactgtgtcagtaaccgaggactctcctgaagcccgagccgagttcccctctggctctgccccccgggagcagaaaaaaaatctcaccttttatctacttctttctctaatcctggtttctgtgggcttcgtggtcacagtgttcggagtaatcatattcaaagtttacaagtggaagcagtctagagacctataccgagccccggtgagctcactgtaccgaacaccagggccctccttgcacgcggacgccgtgcggggaggcctgatgtcgccgcacctttaccatcaggtgtatctcaccacggactcccgccgcagcgacccgctgctgaagaaacctggtgcagccagtccactggccagccgccagaacacgctgcggagctgtgatccggtgttctataggcaggtgttgggtgcagagagcgcccctcccggacagcaagccccgcccaacacggactggcgtttctctcaggcccagagacccggcaccagcggctcccaaaatggcgatgacaccggcacctggcccaacaaccagtttgacacagagatgctgcaagccatgatcttggcgtccgccagtgaagctgctgatgggagctccaccctgggagggggtgccggcaccatgggattgagcgcccgctacggaccccagttcaccctgcagcacgtgcccgactaccgccagaatgtctacatcccaggcagcaatgcacactgaccaacgcagctggcaagcggatggcaaggcccagcaggtggcaatggcaacaagaagaagtcggcaagaaggagaagaagtaacatggaggccaggccaagagccacagggcagcctctccccgaaccagcccagcttctccttacctgcacccaggcctcagagtttcagggctaacccccagaatactggtaggggccaaggcatctcccttggaaacagaaacaagtgccatcacaccatcccttccccaggtgtaatatccaaagcagttccgctgggaaccccatccaatcagtggctgtacccatttgggtagtggggttcatgtagacaccaagaaccatttgccacaccccgtttagttacagctgaaccctccatcttccaaatcaatcaggcccatccatcccatgcctccctcctccccaccccactccaacagttcctctttcccgagtaaggtggttggggtgttgaagtaccaagtaacctacaagcctcctagttctgaaaagttggaagggcatcatgacctcttggcctctcctttgattctcaatcttcccccaaagcatggtttggtgccagccccttcacctccttccagagcccaagatcaatgctcaagttttggaggacatgatcaccatccccatggtactgatgcttgctggatttagggagggcattttgctaccaagcctcttcccaacgccctgggaccagtcttctgttttgtttttcattgtttgagctttccactgcatgccttgacttcccccacctcctcctcaaacaagagactccactgcatgttccaagacagtatggggtggtaagataaggaagggaagtgtgtggatgtggatggtgggggcatggacaaagcttgacacatcaagttatcaaggccttggaggaggctctgtatgtcctcaggggactgacaacatcctccagattccagccataaaccaataactaggctggacccttcccactacataatagggctcagccaggcagccagctttgggctgagctaacaggaccaatggattaactggcatttcagtccaaggaagctcgaagcaggtttaggaccaggtccccttgagaggtcagaggggcctctgtgggtgctgggtactccagaggtgccactggtggaagggtcagcggagccccagcaggaagggtgggccagccaggccattcttagtccctgggttggggaggcagggagctagggcagggaccaaatgaacagaaagtctcagcccaggatggggcttcttcaacaggcccctgccctcctgaagcctcagtccttcaccttgccaggtgccgtttctcttccgtgaaggccactgcccaggtccccagtgcgccccctagtggccatagcctggttaaagttccccagtgcctccttgtgatagaccttcttctcccacccccttctgcccctgggtccccggccatccagcggggctgccagagaaccccagacctgcccttacagtagtgtagcgccccctccctctttcggctggtgtagaatagccagtagtgtagtgcggtgtgcttttacgtgatggcgggtgggcagcgggcggcggcgtccgcgcagccgtctgtccttgatctgcccgcggcggcccgtgttgtgttttgtgctgtgtccagcgctaaggcgaccccctcccccgtactgacttctcctataagcgcttctcttcgcatagtcacgtagctcccaccccaccctcttcctgtgtctcacgcaagttttatactctaatatttatatggctttttttcttcgacaaaaaaataataaaacgtttcttctgaaaaaaaaaaaaaaaaa

SEQ ID NO: 97 - protein, 904 aa
MVPEAWRSGLVSTGRVVGVLLLLGALNKASTVIHYEIPEEREKGFAVGNVVANLGLDLGSLSARRFPVVSGASRRFFEVNRETGEMFVNDRLDREELCGTLPSCTVTLELVVENPLELFSVEVVIQDINDNNPAFPTQEMKLEISEAVAPGTRFPLESAHDPDLGSNSLQTYELSRNEYFALRVQTREDSTKYAELVLERALDREREPSLQLVLTALDGGTPALSASLPIHIKVLDANDNAPVFNQSLYRARVPGGCTSGTRVVQVLATDLDEGPNGEIIYSFGSHNRAGVRQLFALDLVTGMLTIKGRLDFEDTKLHEIYIQAKDKGANPEGAHCKVLVEVVDVNDNAPEITVTSVYSPVPEDASGTVIALLSVTDLDAGENGLVTCEVPPGLPFSLTSSLKNYFTLKTSADLDRETVPEYNLSITARDAGTPSLSALTIVRVQVSDINDNPPQSSQSSYDVYIEENNLPGAPILNLSVWDPDAPQNARLSFFLLEQGAETGLVGRYFTINRDNGIVSSLVPLDYEDRREFELTAHISDGGTPVLATNISVNIFVTDRNDNAPQVLYPRPGGSSVEMLPRGTSAGHLVSRVVGWDADAGHNAWLSYSLFGSPNQSLFAIGLHTGQISTARPVQDTDSPRQTLTVLIKDNGEPSLSTTATLTVSVTEDSPEARAEFPSGSAPREQKKNLTFYLLLSLILVSVGFVVTVFGVIIFKVYKWKQSRDLYRAPVSSLYRTPGPSLHADAVRGGLMSPHLYHQVYLTTDSRRSDPLLKKPGAASPLASRQNTLRSCDPVFYRQVLGAESAPPGQQAPPNTDWRFSQAQRPGTSGSQNGDDTGTWPNNQFDTEMLQAMILASASEAADGSSTLGGGAGTMGLSARYGPQFTLQHVPDYRQNVYIPGSNAH

SEQ ID NO: 98 - protein, 556 aa
DWVIPPINLPENSRGPFPQELVRIRSDRDKNLSLRYTVTGPGADQPPTGIFIINPISGQLSVTKPLDREQIARFHLRAHAVDINGNQVENPIDIVINVIDMNDNRPEFLHQVWNGSVPEGSKPGTYVMTVTAIDADDPNALNGMLRYRILSQAPSTPSPNMFTINNETGDIITVAAGLDREKVQQYTLIIQATDMEGNPTYGLSNTATAVITVTDVNDNPPEFTAMTFYGEVPENRVDIIVANLTVTDKDQPHTPAWNAVTRISGGDPTGRFAIQTDPNSNDGLVTVVKPIDFETNRMFVLTVAAENQVPLAKGIQHPPQSTATVSVTVIDVNENPYFAPNPKIIRQEEGLHAGTMLTTFTAGDPDRYMQQNIRYTKLSDPANWLKIDPVNGQITTIAVLDRESPNVKNNIYNATFLASDNGIPPMSGTGTLQIYLLDINDNAPQVLPQEAETCETPDPNSINITTALDYDIDPNAGPFAYDLPLSPVTIKRNWTITRLNGDFAQLNLKIKFLEAGIYEVPIIITDSGNPPKSNKSILRVRVCQCDFNGDCTDVDR

SEQ ID NO: 99 - protein, 105 aa
EDTVYSFDIPENAQRGYQVGQIVARDADLGQNAQLSYGVVSDWANDVFSLNPQTGMLTLTARLDYEEVQHYILIVQAQDNGQPSLSTTITVYCNVLDLNDNAPIF

SEQ ID NO: 100 - protein, 7 aa
DXDXGXN

SEQ ID NO: 101 - protein, 7 aa
AXDXGXP

SEQ ID NO: 102 - DNA (cDNA), 4650 bp; single-stranded, linear
  CDS 495..4103
cctctattcgacattctctttggattgttttgctataacttgaaatttgggatgtcacaaacgaaactgtcatctgtttccgccaaactgtggttctgctaatctcccaggctggcagcattggagacttgctgacttctttcatcccccactcttttcacctgaaattcctttccttggttttgctctaagtcctatgcttcagtcaggggccaaccaaatctcactgcctcctttttatcatgaagcctttgatcactgatagttctttttatatcttgaaaaatcacccttcccagtacagttaatatttagtatctctactcatcttggcacttactcacagctccataattcagtcgttttcgtacctcttcatggtgatggggagccctttggaggtggtgactgtgctttatactcctcatgatgcttcacatgtggcaggcgtggagtgcccggaggcggccctcctgattctggggcctcccaggatggagcccctgaggcacagcccaggccctggggggcaacggctactgctgccctccatgctgctagcactgctgctcctgctggctccatccccaggccacgccactcgggtagtgtacaaggtgccggaggaacagccacccaacaccctcattgggagcctcgcagccgactatggttttccagatgtggggcacctgtacaagctagaggtgggtgccccgtaccttcgcgtggatggcaagacaggtgacattttcaccaccgagacctccatcgaccgtgaggggctccgtgaatgccagaaccagctccctggtgatccctgcatcctggagtttgaggtatctatcacagacctcgtgcagaatgcgagcccccggctgctagagggccagatagaagtacaagacatcaatgacaacacacccaacttcgcctcaccagtcatcactctggccatccctgagaacaccaacatcggctcactcttccccatcccgctggcttcagaccgtgatgctggtcccaacggtgtggcatcctatgagctgcaggtggcagaggaccaggaggagaagcaaccacagctcattgtgatgggcaacctggaccgtgagcgctgggactcctatgacctcaccatcaaggtgcaggatggcggcagccccccacgcgccacgagtgccctgctgcgtgtcaccgtgcttgacaccaatgacaacgcccccaagtttgagcggccctcctatgaggccgaactatctgagaatagccccataggccactcggtcatccaggtgaaggccaatgactcagaccaaggtgccaatgcagaaatcgaatacacattccaccaggcgcccgaagttgtgaggcgtcttcttcgactggacaggaacactggacttatcactgttcagggcccggtggaccgtgaggacctaagcaccctgcgcttctcagtgcttgctaaggaccgaggcaccaaccccaagagtgcccgtgcccaggtggttgtgaccgtgaaggacatgaatgacaatgcccccaccattgagatccggggcatagggctagtgactcatcaagatgggatggctaacatctcagaggatgtggcagaggagacagctgtggccctggtgcaggtgtctgaccgagatgagggagagaatgcagctgtcacctgtgtggtggcaggtgatgtgcccttccagctgcgccaggccagtgagacaggcagtgacagcaagaagaagtatttcctgcagactaccaccccgctagactacgagaaggtcaaagactacaccattgagattgtggctgtggactctggcaaccccccactctccagcactaactccctcaaggtgcaggtggtggacgtcaatgacaacgcacctgtcttcactcagagtgtcactgaggtcgccttcccggaaaacaacaagcctggtgaagtgattgctgagatcactgccagtgatgctgactctggctctaatgctgagctggtttactctctggagcctgagccggctgctaagggcctcttcaccatctcacccgagactggagagatccaggtgaagacatctctggatcgggaacagcgggagagctatgagttgaaggtggtggcagctgaccggggcagtcctagcctccagggcacagccactgtccttgtcaatgtgctggactgcaatgacaatgaccccaaatttatgctgagtggctacaacttctcagtgatggagaacatgccagcactgagtccagtgggcatggtgactgtcattgatggagacaagggggagaatgcccaggtgcagctctcagtggagcaggacaacggtgactttgttatccagaatggcacaggcaccatcctatccagcctgagctttgatcgagagcaacaaagcacctacaccttccagctgaaggcagtggatggtggcgtcccacctcgctcagcttacgttggtgtcaccatcaatgtgctggacgagaatgacaacgcaccctatatcactgccccttctaacacctctcacaagctgctgaccccccagacacgtcttggtgagacggtcagccaggtggcagccgaggactttgactctggtgtcaatgccgagctgatctacagcattgcaggtggcaacccttatggactcttccagattgggtcacattcaggtgccatcaccctggagaaggagattgagcggcgccaccatgggctacaccgcctggtggtgaaggtcagtgaccgcggcaagcccccacgctatggcacagccttggtccatctttatgtcaatgagactctggccaaccgcacgctgctggagaccctcctgggccacagcctggacacgccgctggatattgacattgctggggatccagaatatgagcgctccaagcagcgtggcaacattctctttggtgtggtggctggtgtggtggccgtggccttgctcatcgccctggcggttcttgtgcgctactgcagacagcgggaggccaaaagtggttaccaggctggtaagaaggagaccaaggacctgtatgcccccaagcccagtggcaaggcctccaagggaaacaaaagcaaaggcaagaagagcaagtccccaaagcccgtgaagccagtggaggacgaggatgaggccgggctgcagaagtccctcaagttcaacctgatgagcgatgcccctggggacagtccccgcatccacctgcccctcaactacccaccaggcagccctgacctgggccgccactatcgctctaactccccactgccttccatccagctgcagccccagtcaccctcagcctccaagaagcaccaggtggtacaggacctgccacctgcaaacacattcgtgggcaccggggacaccacgtccacgggctctgagcagtactccgactacagctaccgcaccaacccccccaaataccccagcaagcagttacctcaccgccgcgtcaccttctcggccaccagccaggcccaggagctgcaggacccatcccagcacagttactatgacagtggcctggaggagtctgagacgccgtccagcaagtcatcctcagggcctcgactcggtcccctggccctgcctgaggatcactatgagcgcaccacccctgatggcagcataggagagatggagcaccccgagaatgaccttcgccctttgcctgatgtcgccatgacaggcacatgtacccgggagtgcagtgagtttggccactctgacacatgctggatgcctggccagtcatctcccagccgccggaccaagagcagcgccctcaaactctccaccttcatgccttaccaggaccgaggagggcaggagcctgcgggcgccggcagccccagccccccggaagaccggaacaccaaaacggcccccgtgcgcctcctgccctcctacagtgccttctcccacagtagccatgattcctgcaaggactcggccaccttggaggaaatccccctgacccagacctcggacttcccacccgcagccacaccggcatctgcccagacggccaagcgcgagatctacctgtgagccccctactggccggcccccctcccccagcgccggccagctcccaaatgcccattccagggcctcactctccaccccttcagcgtggacttcctgccagggcccaagtgggggtatcactgacctcatgaccacgctggcccttctcccatgcagggtccaggtcctctcccctcatttccatctcccagcccaggggccccttcccctttatggggcttcccccagctgatgcccaagagggctcctctgcaatgactgggctccttcccttgacttccagggagcaccccctcgatttgggcagatggtggagtcaagggtgggcagcgtacttctaactcattgtttccctcatggccgaccagggcggggatagcatgcccaattttagccctgaagcagggctgaactggggagcccctttccctgggagctcccagaggaaactcttgaccaccagtggctccctgaagggcttttgttaccaaaggtggggtagggacgggggtgggagtggagcggaggccttgttttcccgtgg

SEQ ID NO: 103 - protein, 1203 aa
MEPLRHSPGPGGQRLLLPSMLLALLLLLAPSPGHATRVVYKVPEEQPPNTLIGSLAADYGFPDVGHLYKLEVGAPYLRVDGKTGDIFTTETSIDREGLRECQNQLPGDPCILEFEVSITDLVQNASPRLLEGQIEVQDINDNTPNFASPVITLAIPENTNIGSLFPIPLASDRDAGPNGVASYELQVAEDQEEKQPQLIVMGNLDRERWDSYDLTIKVQDGGSPPRATSALLRVTVLDTNDNAPKFERPSYEAELSENSPIGHSVIQVKANDSDQGANAEIEYTFHQAPEVVRRLLRLDRNTGLITVQGPVDREDLSTLRFSVLAKDRGTNPKSARAQVVVTVKDMNDNAPTIEIRGIGLVTHQDGMANISEDVAEETAVALVQVSDRDEGENAAVTCVVAGDVPFQLRQASETGSDSKKKYFLQTTTPLDYEKVKDYTIEIVAVDSGNPPLSSTNSLKVQVVDVNDNAPVFTQSVTEVAFPENNKPGEVIAEITASDADSGSNAELVYSLEPEPAAKGLFTISPETGEIQVKTSLDREQRESYELKVVAADRGSPSLQGTATVLVNVLDCNDNDPKFMLSGYNFSVMENMPALSPVGMVTVIDGDKGENAQVQLSVEQDNGDFVIQNGTGTILSSLSFDREQQSTYTFQLKAVDGGVPPRSAYVGVTINVLDENDNAPYITAPSNTSHKLLTPQTRLGETVSQVAAEDFDSGVNAELIYSIAGGNPYGLFQIGSHSGAITLEKEIERRHHGLHRLVVKVSDRGKPPRYGTALVHLYVNETLANRTLLETLLGHSLDTPLDIDIAGDPEYERSKQRGNILFGVVAGVVAVALLIALAVLVRYCRQREAKSGYQAGKKETKDLYAPKPSGKASKGNKSKGKKSKSPKPVKPVEDEDEAGLQKSLKFNLMSDAPGDSPRIHLPLNYPPGSPDLGRHYRSNSPLPSIQLQPQSPSASKKHQVVQDLPPANTFVGTGDTTSTGSEQYSDYSYRTNPPKYPSKQLPHRRVTFSATSQAQELQDPSQHSYYDSGLEESETPSSKSSSGPRLGPLALPEDHYERTTPDGSIGEMEHPENDLRPLPDVAMTGTCTRECSEFGHSDTCWMPGQSSPSRRTKSSALKLSTFMPYQDRGGQEPAGAGSPSPPEDRNTKTAPVRLLPSYSAFSHSSHDSCKDSATLEEIPLTQTSDFPPAATPASAQTAKREIYL

SEQ ID NO: 104 - DNA (cDNA), 2789 bp; single-stranded, linear
  CDS 115..2622
cgaaagccatgtcggactcgtcgcccagcgcccaagcgctaacccgctgaaagtttctcagcgaaatctcagggacgatctggaccccgctgagaggaactgcttttgagtgagatggtcccagaggcctggaggagcggactggtaagcaccgggagggtagtgggagttttgcttctgcttggtgccttgaacaaggcttccacggtcattcactatgagatcccggaggaaagagagaagggtttcgctgtgggcaacgtggtcgcgaaccttggtttggatctcggtagcctctcagcccgcaggttcccggtggtgtctggagctagccgaagattctttgaggtgaaccgggagaccggagagatgtttgtgaacgaccgtctggatcgagaggagctgtgtgggacactgccctcttgcactgtaactctggagttggtagtggagaacccgctggagctgttcagcgtggaagtggtgatccaggacatcaacgacaacaatcctgctttccctacccaggaaatgaaattggagattagcgaggccgtggctccggggacgcgctttccgctcgagagcgcgcacgatcccgatctgggaagcaactctttacaaacctatgagctgagccgaaatgaatactttgcgcttcgcgtgcagacgcgggaggacagcaccaagtacgcggagctggtgttggagcgcgccctggaccgagaacgggagcctagtctccagttagtgctgacggcgttggacggagggaccccagctctctccgccagcctgcctattcacatcaaggtgctggacgcgaatgacaatgcgcctgtcttcaaccagtccttgtaccgggcgcgcgttcctggaggatgcacctccggcacgcgcgtggtacaagtccttgcaacggatctggatgaaggccccaacggtgaaattatttactccttcggcagccacaaccgcgccggcgtgcggcaactattcgccttagaccttgtaaccgggatgctgacaatcaagggtcggctggacttcgaggacaccaaactccatgagatttacatccaggccaaagacaagggcgccaatcccgaaggagcacattgcaaagtgttggtggaggttgtggatgtgaatgacaacgccccggagatcacagtcacctccgtgtacagcccagtacccgaggatgcctctgggactgtcatcgctttgctcagtgtgactgacctggatgctggcgagaacgggctggtgacctgcgaagttccaccgggtctccctttcagccttacttcttccctcaagaattacttcactttgaaaaccagtgcagacctggatcgggagactgtgccagaatacaacctcagcatcaccgcccgagacgccggaaccccttccctctcagcccttacaatagtgcgtgttcaagtgtccgacatcaatgacaaccctccacaatcttctcaatcttcctacgacgtttacattgaagaaaacaacctccccggggctccaatactaaacctaagtgtctgggaccccgacgccccgcagaatgctcggctttctttctttctcttggagcaaggagctgaaaccgggctagtgggtcgctatttcacaataaatcgtgacaatggcatagtgtcatccttagtgcccctagactatgaggatcggcgggaatttgaattaacagctcatatcagcgatgggggcaccccggtcctagccaccaacatcagcgtgaacatatttgtcactgatcgcaatgacaatgccccccaggtcctatatcctcggccaggtgggagctcggtggagatgctgcctcgaggtacctcagctggccacctagtgtcacgggtggtaggctgggacgcggatgcagggcacaatgcctggctctcctacagtctctttggatcccctaaccagagcctttttgccatagggctgcacactggtcaaatcagtactgcccgtccagtccaagacacagattcacccaggcagactctcactgtcttgatcaaagacaatggggagccttcgctctccaccactgctaccctcactgtgtcagtaaccgaggactctcctgaagcccgagccgagttcccctctggctctgccccccgggagcagaaaaaaaatctcaccttttatctacttctttctctaatcctggtttctgtgggcttcgtggtcacagtgttcggagtaatcatattcaaagtttacaagtggaagcagtctagagacctataccgagccccggtgagctcactgtaccgaacaccagggccctccttgcacgcggacgccgtgcggggaggcctgatgtcgccgcacctttaccatcaggtgtatctcaccacggactcccgccgcagcgacccgctgctgaagaaacctggtgcagccagtccactggccagccgccagaacacgctgcggagctgtgatccggtgttctataggcaggtgttgggtgcagagagcgcccctcccggacaggtaaggtttagcaagtcatgcttgaccctgttagtgcctttttattcctacatcatattgagaaggctggagctgtttttttagtgatgaagatgttttcctggtgatgcattcacactttcaactggctcttcctagatcaaagttagtgcctttgtgagatggtggcctgccagagtgtggtttgtggtcccatttcagggggaagatacttgactcatctgtggacctaattcacatcctcagcg

SEQ ID NO: 105 - protein, 836 aa
MVPEAWRSGLVSTGRVVGVLLLLGALNKASTVIHYEIPEEREKGFAVGNVVANLGLDLGSLSARRFPVVSGASRRFFEVNRETGEMFVNDRLDREELCGTLPSCTVTLELVVENPLELFSVEVVIQDINDNNPAFPTQEMKLEISEAVAPGTRFPLESAHDPDLGSNSLQTYELSRNEYFALRVQTREDSTKYAELVLERALDREREPSLQLVLTALDGGTPALSASLPIHIKVLDANDNAPVFNQSLYRARVPGGCTSGTRVVQVLATDLDEGPNGEIIYSFGSHNRAGVRQLFALDLVTGMLTIKGRLDFEDTKLHEIYIQAKDKGANPEGAHCKVLVEVVDVNDNAPEITVTSVYSPVPEDASGTVIALLSVTDLDAGENGLVTCEVPPGLPFSLTSSLKNYFTLKTSADLDRETVPEYNLSITARDAGTPSLSALTIVRVQVSDINDNPPQSSQSSYDVYIEENNLPGAPILNLSVWDPDAPQNARLSFFLLEQGAETGLVGRYFTINRDNGIVSSLVPLDYEDRREFELTAHISDGGTPVLATNISVNIFVTDRNDNAPQVLYPRPGGSSVEMLPRGTSAGHLVSRVVGWDADAGHNAWLSYSLFGSPNQSLFAIGLHTGQISTARPVQDTDSPRQTLTVLIKDNGEPSLSTTATLTVSVTEDSPEARAEFPSGSAPREQKKNLTFYLLLSLILVSVGFVVTVFGVIIFKVYKWKQSRDLYRAPVSSLYRTPGPSLHADAVRGGLMSPHLYHQVYLTTDSRRSDPLLKKPGAASPLASRQNTLRSCDPVFYRQVLGAESAPPGQVRFSKSCLTLLVPFYSYIILRRLELFF

SEQ ID NO: 106 - DNA (cDNA), 2751 bp; single-stranded, linear
  CDS 115..2160
cgaaagccatgtcggactcgtcgcccagcgcccaagcgctaacccgctgaaagtttctcagcgaaatctcagggacgatctggaccccgctgagaggaactgcttttgagtgagatggtcccagaggcctggaggagcggactggtaagcaccgggagggtagtgggagttttgcttctgcttggtgccttgaacaaggcttccacggtcattcactatgagatcccggaggaaagagagaagggtttcgctgtgggcaacgtggtcgcgaaccttggtttggatctcggtagcctctcagcccgcaggttcccggtggtgtctggagctagccgaagattctttgaggtgaaccgggagaccggagagatgtttgtgaacgaccgtctggatcgagaggagctgtgtgggacactgccctcttgcactgtaactctggagttggtagtggagaacccgctggagctgttcagcgtggaagtggtgatccaggacatcaacgacaacaatcctgctttccctacccaggaaatgaaattggagattagcgaggccgtggctccggggacgcgctttccgctcgagagcgcgcacgatcccgatctgggaagcaactctttacaaacctatgagctgagccgaaatgaatactttgcgcttcgcgtgcagacgcgggaggacagcaccaagtacgcggagctggtgttggagcgcgccctggaccgagaacgggagcctagtctccagttagtgctgacggcgttggacggagggaccccagctctctccgccagcctgcctattcacatcaaggtgctggacgcgaatgacaatgcgcctgtcttcaaccagtccttgtaccgggcgcgcgttcctggaggatgcacctccggcacgcgcgtggtacaagtccttgcaacggatctggatgaaggccccaacggtgaaattatttactccttcggcagccacaaccgcgccggcgtgcggcaactattcgccttagaccttgtaaccgggatgctgacaatcaagggtcggctggacttcgaggacaccaaactccatgagatttacatccaggccaaagacaagggcgccaatcccgaaggagcacattgcaaagtgttggtggaggttgtggatgtgaatgacaacgccccggagatcacagtcacctccgtgtacagcccagtacccgaggatgcctctgggactgtcatcgctttgctcagtgtgactgacctggatgctggcgagaacgggctggtgacctgcgaagttccaccgggtctccctttcagccttacttcttccctcaagaattacttcactttgaaaaccagtgcagacctggatcgggagactgtgccagaatacaacctcagcatcaccgcccgagacgccggaaccccttccctctcagcccttacaatagtgcgtgttcaagtgtccgacatcaatgacaaccctccacaatcttctcaatcttcctacgacgtttacattgaagaaaacaacctccccggggctccaatactaaacctaagtgtctgggaccccgacgccccgcagaatgctcggctttctttctttctcttggagcaaggagctgaaaccgggctagtgggtcgctatttcacaataaatcgtgacaatggcatagtgtcatccttagtgcccctagactatgaggatcggcgggaatttgaattaacagctcatatcagcgatgggggcaccccggtcctagccaccaacatcagcgtgaacatatttgtcactgatcgcaatgacaatgccccccaggtcctatatcctcggccaggtgggagctcggtggagatgctgcctcgaggtacctcagctggccacctagtgtcacgggtggtaggctgggacgcggatgcagggcacaatgcctggctctcctacagtctctttggatcccctaaccagagcctttttgccatagggctgcacactggtcaaatcagtactgcccgtccagtccaagacacagattcacccaggcagactctcactgtcttgatcaaagacaatggggagccttcgctctccaccactgctaccctcactgtgtcagtaaccgaggactctcctgaagcccgagccgagttcccctctggctctgccagttaaaccttctttaattatggattagccattaacatttttgaaacgtggaccatttaacctcggcctaccccctccaactgtcctggtgatgagttcattagctaagttaaattaattgaactttgatctaaaccaaaacaaatcaggaaaataaagctgtaaaggaacttatcaagcattccaaaaccaactagaaattacttgaagtttcgagtgagcattgcctgtgccagtattcttcattataggattataaactcgtttttttcccaaagcgcatgtctacgccaggcagaggagtaattattcagccaatttcatggatgtaacgatggatataaataattgatagcacctagaggcttccagtttgggtggaaggctaaaagtagaggggaactcactcacttgagaaatgatatttaagtgaataaatagttctcttctatgaaactattactatttagttctctggaaaacttaagtgtattaatgattagaacatcaaatcctaagtaaagaaatgacattttaaatataaaaagccaaactttaaataaatcatagagacctcagacataatataggaaa

SEQ ID NO: 107 - protein, 682 aa
MVPEAWRSGLVSTGRVVGVLLLLGALNKASTVIHYEIPEEREKGFAVGNVVANLGLDLGSLSARRFPVVSGASRRFFEVNRETGEMFVNDRLDREELCGTLPSCTVTLELVVENPLELFSVEVVIQDINDNNPAFPTQEMKLEISEAVAPGTRFPLESAHDPDLGSNSLQTYELSRNEYFALRVQTREDSTKYAELVLERALDREREPSLQLVLTALDGGTPALSASLPIHIKVLDANDNAPVFNQSLYRARVPGGCTSGTRVVQVLATDLDEGPNGEIIYSFGSHNRAGVRQLFALDLVTGMLTIKGRLDFEDTKLHEIYIQAKDKGANPEGAHCKVLVEVVDVNDNAPEITVTSVYSPVPEDASGTVIALLSVTDLDAGENGLVTCEVPPGLPFSLTSSLKNYFTLKTSADLDRETVPEYNLSITARDAGTPSLSALTIVRVQVSDINDNPPQSSQSSYDVYIEENNLPGAPILNLSVWDPDAPQNARLSFFLLEQGAETGLVGRYFTINRDNGIVSSLVPLDYEDRREFELTAHISDGGTPVLATNISVNIFVTDRNDNAPQVLYPRPGGSSVEMLPRGTSAGHLVSRVVGWDADAGHNAWLSYSLFGSPNQSLFAIGLHTGQISTARPVQDTDSPRQTLTVLIKDNGEPSLSTTATLTVSVTEDSPEARAEFPSGSAS

SEQ ID NO: 108 - DNA (cDNA), 2831 bp; single-stranded, linear
gaattcggcacgaggctgaactgagggtgacggacataaacgactattctccagtgttcagtgaaagagaaatgatactgaggataccagaaaacagtgctcggggaaatacattccctttaaacaatgctctggactcagacgtagatatcaacaatatccagacctataggctcagctcaaactctcatttcctggttgtaacccgcaaccgcagtgatggcaggaagtacccagagctggtgctggagaaagaactggatcgagaggaggaacctgagctgaggttaacgctgacagctttggatggtggctctcctccccggtctgggacgacacaggtcctcattgaagtagtggacaccaacgataatgcacccgagtttcagcagccaacataccaagtgcaaactcccgagaacagtcccaccggctctctggtactcacagtctcagccaatgacttagacagtggagactatgggaaagtcttgtacgcactttcgcaaccctcagaagatattagcaaaacattcgaggtaaaccctgtaaccggggaaattcgcctacgaaaagaggtgaattttgaaactattccttcgtatgaagtggttatcaaggggacggacgggggaggtctctcaggaaaatgcactctgttactgcaggtggtggacgtgaatgacaatgccccagaagtgatgctatctgcgctaaccaacccagtcccagaaaattcccccgatgaggtagtggctgttttcagtgttagagatcctgactctgggaacaacggaaaagtgattgcatccatcgaggaagacctgccctttcttctaaaatcttcaggaaagaacttttacactttagtaaccaagggagcacttgacagggaagaaagagagcaattgaacatcaccatcacagtcactgacctgggcatacccaggctcaccacccaacacaccataacagtgcaggtggcagacatcaacgacaatgccccctccttcacccaaacctcctacaccatgtttgtccgcgagaacaacagccccgccctgcacataggcaccatcagcgccacagactcagactcaggatccaatgcccacatcacctactcgctgctaccgccccaagacccacagctggccctcgactcgctcatctccatcaatgtagacaacgggcagctgttcgcgctcagggcgctagactatgaggctctgcagggcttcgagttccatgtgggcgccacagaccaaggctcgcccgcgctcagcagccaggctctggtgcacgtggtggtgttggacgacaatgacaatgcgcccttcgtgctctacccgctgcaaaacgcctctgcacccttcactgagctgctgcccagggcggcagagcctggatacctggttaccaaggtggtagctgtggaccgcgactctggccagaatgcctggctgtcattccagctgctcaaggccacggagcccgggctgttcaacgtatgggcgcacaatggcgaggtacgcacctccaggctgctgagcgagcgcgacgcacccaagcacaagctgctgctgttggtcaaggacaatggagatcctccacgctctgccagtgttactctgcacgtgctagtggtggatgccttctctcagccctacctgcctctgccagaggtggcgcacgaccctgcacaagaagaagatgcgctaacactctacctggtcatagctttggcatctgtgtcttctctcttcctcttgtctgtgctgctgttcgtgggggtgaggctctgcaggagggccagggcagcctctctgagtgcctattctgtgcctgaaggccactttcctggccagctggtggatgtcagaggtatggggaccctgtcccagagctaccagtatgatgtatgtctgatgggggattcttctgggaccagcgaatttaacttcttaaagccagttctgcctagctctctgcaccagtgctctgggaaagaaatagaggaaaattccacactccagaatagttttgggtttcatcattaatagaaaactactttacagatatttaattccaaatatcatcttgttgattaactaaagtctgttcacatgtagctagctagcaacgattttaatgttcactttacccatcttttttcagggtcatgtctaaagctacaagtttgnctttacttatacttgtcgcacagaatnnnnnnnnnntggtgtataagtcacagtcatgggatactggcacaagatggcagcttgattgctcagttatggctgcaaaggggngcttgagtttagggaatgtgttagagctggaataagttttctgagaaatgtgtaagacaaatttcttttgcacattccctgtgttcctgtacccctgtttccagaactacgaaatgtgtcatcagaaggcatgctcacattttcccctttgtttgcgtgacccgggtgccagaaattaaataaaattagcatggagttcaatgcagcattaaaacaaagttacttctacaaaccttttattcgacggttaaaattgtaacttccccacccatgaggctggctgtaagaaccagtatgaatgggtgtctatcgcaaccttattttcaaaaatcaaacaaaaggagaaatgagagaccaaacaacacgctacaggaaagatttcataaggatgtatgtatggacacaaaaactgggatacagacattttaaatctgttggtaccacatggtggcgctgcaggctaaagaaatgcaagggaaattaaaaagaggctgagctagaagtcaaaaaaaaaaa

SEQ ID NO: 109 - DNA (cDNA), 3353 bp; single-stranded, linear
  CDS 763..3123
gtatttttccacagtttaaaattttcataaaatcataactctctgactttatgtagaaaggataccacactggaattaacgtgtagctttttcttgatgtaatccaaccaatgggagcacaattctggtacataggctgtctagaatttgaaagaaattaaagaattcattttgttttgctgataaatttttaagaaatcacgtggctttatgttattattattacaagatgactgatcactattatgtcttctttcacttctcaatttccctcagaacactacacccagactacaggctctggagggtggggaccatgtctgggttgtttactgatgtatttcataatttggcacatagagaccaataatactcctttaaatgaagaaattaataattaccattgcgtgatattgtgattacatcatttcctcccaatttccaaactcctaatagaatagagaatagatcaattgtagcaattcgtttcgaagcaaagacaacgcatggtggcgctgcaggctaaggcttcaaaaaaaggaaaaggaaaaagcccatgaaatgctactagctacttcagacctctttcagcctaagaggaaagcctgttagcagagcacggaccagtgtctccggagaatgctattctcctacatttccgaacaggttatcaacgcacagatcgatcactgcctctgtcccatcgctccctgaagtagctctgactccggttccttgaaaggggcgtgtacagaagtaaagatggagcctgcaggggagcgctttcccgaacaaaggcaagtcctgattctccttcttttactggaagtgactctggcaggctgggaaccccgtcgctattctgtgatggaggaaacagagagaggttcttttgtagccaacctggccaatgacctagggctgggagtgggggagctagccgagcggggagcccgggtagtttctgaggataacgaacaaggcttgcagcttgatctgcagaccgggcagttgatattaaatgagaagctggaccgggagaagctgtgtggccctactgagccctgtataatgcatttccaagtgttactgaaaaaacctttggaagtatttcgagctgaactactagtgacagacataaacgatcattctcctgagtttcctgaaagagaaatgaccctgaaaatcccagaaactagctcccttgggactgtgtttcctctgaaaaaagctcgggacttggacgtgggcagcaataatgttcaaaactacaatatttctcccaattctcatttccatgtttccactcgcacccgaggggatggcaggaaatacccagagctggtgctggacacagaactggatcgcgaggagcaggccgagctcagattaaccttgacagcggtggacggtggctctccaccccgatctggcaccgtccagatcctcatcttggtcttggacgccaatgacaatgccccggagtttgtgcaggcgctctacgaggtgcaggtcccagagaacagcccagtaggctccctagttgtcaaggtctctgctagggatttagacactgggacaaatggagagatatcatactccctttattacagctctcaggagatagacaaaccttttgagctaagcagcctttcaggagaaattcgactaattaaaaaactagattttgagacaatgtcttcatatgatctagatatagaggcatctgatggcgggggactttctggaaaatgctctgtctctgttaaggtgctggatgttaacgataacttcccggaactaagtatttcatcacttaccagccctattcccgagaattctccagagacagaagtggccctgtttaggattagagaccgagactctggagaaaatggaaaaatgatttgctcaattcaggatgatgttccttttaagctaaaaccttctgttgagaatttctacaggctggtaacagaaggggcgctggacagagagaccagagccgagtacaacatcaccatcaccatcacagacttggggactccaaggctgaaaaccgagcagagcataaccgtgctggtgtcggacgtcaatgacaacgcccccgccttcacccaaacctcctacaccctgttcgtccgcgagaacaacagccccgccctgcacatcggcagtgtcagcgccacagacagagactcgggcaccaacgcccaggtcacctactcgctgctgccgccccaggacccgcacctgcccctaacctccctggtctccattaacacggacaacggccacctgttcgctctccagtcgctggactacgaggccctgcaggctttcgagttccgcgtgggcgccacagaccgcggcttcccggcgctgagcagcgaggcgctggtgcgagtgctggtgctggacgccaacgacaactcgcccttcgtgctgtacccgctgcagaacggctccgcgccctgcaccgagctggtgccccgggcggccgagccgggctacctggtgaccaaggtggtggcggtggacggcgactcgggccagaacgcctggctgtcgtaccagctgctcaaggccacggagcccgggctgttcggcgtgtgggcgcacaatggcgaggtgcgcaccgccaggctgctgagcgagcgcgacgtggccaagcacaggctagtggtgctggtcaaggacaatggcgagcctccgcgctcggccacagccacgctgcaagtgctcctggtggacggcttctctcagccctacctgccgctcccagaggcggccccggcccaagcccaggccgactcgcttaccgtctacctggtggtggcattggcctcggtgtcttcgctcttcctcttctcggtgttcctgttcgtggcagtgcggctgtgcaggaggagcagggcggcctcagtgggtcgctgctcggtgcccgagggcccctttccagggcatctggtggacgtgagcggcaccgggaccctttcccagagctaccagtacgaggtgtgtctgacgggaggctctgaaagtaatgatttcaagttcttgaagcctatattcccaaatattgtaagccaggactctaggaggaaatcagaatttctagaataatgtaggtatctgtagctttccgaccgtctgttaattttgtcttcctcacttttcaccttagttttttttaaccctttagtaatcttgaattctacttttttttaaatttctactgttgtctttagtaatgttactcatttcctttgtctgattgttagttttcaaattattgtattattataaatattttatatcaggaaagttcatatttctgaataaattaatag

SEQ ID NO: 110 - protein, 787 aa
MEPAGERFPEQRQVLILLLLLEVTLAGWEPRRYSVMEETERGSFVANLANDLGLGVGELAERGARVVSEDNEQGLQLDLQTGQLILNEKLDREKLCGPTEPCIMHFQVLLKKPLEVFRAELLVTDINDHSPEFPEREMTLKIPETSSLGTVFPLKKARDLDVGSNNVQNYNISPNSHFHVSTRTRGDGRKYPELVLDTELDREEQAELRLTLTAVDGGSPPRSGTVQILILVLDANDNAPEFVQALYEVQVPENSPVGSLVVKVSARDLDTGTNGEISYSLYYSSQEIDKPFELSSLSGEIRLIKKLDFETMSSYDLDIEASDGGGLSGKCSVSVKVLDVNDNFPELSISSLTSPIPENSPETEVALFRIRDRDSGENGKMICSIQDDVPFKLKPSVENFYRLVTEGALDRETRAEYNITITITDLGTPRLKTEQSITVLVSDVNDNAPAFTQTSYTLFVRENNSPALHIGSVSATDRDSGTNAQVTYSLLPPQDPHLPLTSLVSINTDNGHLFALQSLDYEALQAFEFRVGATDRGFPALSSEALVRVLVLDANDNSPFVLYPLQNGSAPCTELVPRAAEPGYLVTKVVAVDGDSGQNAWLSYQLLKATEPGLFGVWAHNGEVRTARLLSERDVAKHRLVVLVKDNGEPPRSATATLQVLLVDGFSQPYLPLPEAAPAQAQADSLTVYLVVALASVSSLFLFSVFLFVAVRLCRRSRAASVGRCSVPEGPFPGHLVDVSGTGTLSQSYQYEVCLTGGSESNDFKFLKPIFPNIVSQDSRRKSEFLE

SEQ ID NO: 111 - DNA (cDNA), 3033 bp; single-stranded, linear
  CDS 138..2528
gtgattggacgtgtttttgtgactatttgggaagaagacaccttcctaatcagatttactccaatatcttcccggaccctcatgagtggattgcaattgacttgaagaagcagcaccctcaggactgaatctgaacaatggagacagcactagcaaaaataccacagcaaaggcaagtcttttttcttactatattgtcgttattgtggaagtctagctctgaggccattagatattccatgccagaagaaacagagagtggctatatggtggctaacctggcgaaagatctggggatcagggttggagaactgtcctctagaggagctcaaatccattacaaaggaaacaaagaacttttgcagctggatgcagagactgggaatttgttcttaaaggaaaaactagacagagaactgctgtgtggagagacagaaccctgtgtgctgaacttccagatcatactggaaaaccctatgcagttcttccaaactgaactgcagctcacagatataaacgaccattctccagagttccccaacaagaaaatgcttctaacaattcctgagagtgcccatccagggactgtgtttcctctgaaggcagctcgggactctgacatagggagcaacgctgttcagaactacacagtcaatcccaacctccatttccacgtcgttactcacagtcgcacagatggcaggaaatacccagagctggtgctggacagagccctggatagggaggagcagcctgagctcactttaatcctcactgctctggatggtggagctccttccaggtcaggaaccaccacagttcacatagaagttgtggacatcaatgataactccccccagtttgtacagtcactctataaggtgcaagttcctgagaataatcccctcaatgcctttgttgtcacggtctctgccacggatttagatgctggggtatatggcaatgtgacctattctctgtttcaagggtatggggtatttcaaccatttgtaatagacgaaatcactggagaaatccatctgagcaaagagctggattttgaggaaattagcaatcataacatagaaatcgcagccacagatggaggaggcctttcaggaaaatgcactgtggctgtacaggtgttggatgtgaatgacaacgccccagagttgacaattaggaagctcacagtcctggtcccagaaaattccgcagagactgtagttgctgtttttagtgtttctgattctgattcgggggacaatggaaggatggtgtgttctattccgaacaatatcccatttctcctgaaacccacatttgagaattattacacgttagtgactgaggggccacttgatagagagaacagagctgagtacaacatcaccatcacggtctcagatctgggcacacccaggctcacaacccagcacaccataacagtgcaagtgtccgacatcaacgacaacgcccctgccttcacccaaacctcctacaccatgtttgtccacgagaacaacagccccgccctgcacataggcaccatcagtgccacagactcagactcaggctccaatgcccacatcacctactcgctgctgccgcctgatgacccgcagctggccctcgactcactcatctccatcaatgttgacaatgggcagctgttcgcgctcagagctctagactatgaggcactgcagtccttcgagttctacgtgggcgctacagatggaggctcacccgcgctcagcagccagactctggtgcggatggtggtgctggatgacaatgacaatgcccccttcgtgctctacccactgcagaatgcctcagcaccctgtactgagctactgcctagggcagcagagcccggctacctgatcaccaaagtggtggctgtggatcgcgactctggacagaatgcttggctgtcgttccagctacttaaagctacagagccagggctgttcagtgtatgggcacacaatggtgaagtgcgcaccactaggctgctgagtgagcgagatgctcagaagcacaagctactgctgctggtcaaggacaatggcgatcctctgcgctctgccaatgtcactcttcacgtgctagtggtggatggcttctcgcagccttacctaccattggctgaggtggcacaggattccatgcaagataattacgacgttctcacactgtacctagtcattgccttggcatctgtatcttctctcttcctcttgtctgtagtgctgtttgtgggggtgaggctgtgcaggagggccagggaggcctccttgggtgactactctgtgcctgagggacactttcctagccacttggtggatgtcagcggtgccgggaccctgtcccagagttatcaatatgaggtgtgtcttaatggaggtactagaacaaatgagtttaactttcttaaaccattgtttcctatccttccgacccaggctgctgctgctgaagaaagagaaaacgctgttgtgcacaatagcgttggattctattagagcactgattttgaagtggtggttacctcatttttccttaactatccctgatgtagaatggtgtagtgccgtgaatcaactcctgagatatatgttcattttatcctttgttttgaatcaaactattcagatgtgatcctactctagagaatttggttctactccattgtgtttgtttagatttctacgccataccagtgcatgctgggttgtttttttttttacaattattataactttgctttggaggggaactcatattcgctgtaacgaattggaaccactttcattgttagagatgccttgctttgttgtgttatttcagacagggtcttaaattgtagccctgggtgacctgaaatgactatgtacagactgactttgaatttgtggcagtccatctgcctctgttgtcctatgttgggattgtgagcatgcatgagtaggctcagctgtggtgagcgaccttaataaaaatcaaatactaaaaaaaaaaaaaaaa

SEQ ID NO: 112 - protein, 797 aa
METALAKIPQQRQVFFLTILSLLWKSSSEAIRYSMPEETESGYMVANLAKDLGIRVGELSSRGAQIHYKGNKELLQLDAETGNLFLKEKLDRELLCGETEPCVLNFQIILENPMQFFQTELQLTDINDHSPEFPNKKMLLTIPESAHPGTVFPLKAARDSDIGSNAVQNYTVNPNLHFHVVTHSRTDGRKYPELVLDRALDREEQPELTLILTALDGGAPSRSGTTTVHIEVVDINDNSPQFVQSLYKVQVPENNPLNAFVVTVSATDLDAGVYGNVTYSLFQGYGVFQPFVIDEITGEIHLSKELDFEEISNHNIEIAATDGGGLSGKCTVAVQVLDVNDNAPELTIRKLTVLVPENSAETVVAVFSVSDSDSGDNGRMVCSIPNNIPFLLKPTFENYYTLVTEGPLDRENRAEYNITITVSDLGTPRLTTQHTITVQVSDINDNAPAFTQTSYTMFVHENNSPALHIGTISATDSDSGSNAHITYSLLPPDDPQLALDSLISINVDNGQLFALRALDYEALQSFEFYVGATDGGSPALSSQTLVRMVVLDDNDNAPFVLYPLQNASAPCTELLPRAAEPGYLITKVVAVDRDSGQNAWLSFQLLKATEPGLFSVWAHNGEVRTTRLLSERDAQKHKLLLLVKDNGDPLRSANVTLHVLVVDGFSQPYLPLAEVAQDSMQDNYDVLTLYLVIALASVSSLFLLSVVLFVGVRLCRRAREASLGDYSVPEGHFPSHLVDVSGAGTLSQSYQYEVCLNGGTRTNEFNFLKPLFPILPTQAAAAEERENAVVHNSVGFY

SEQ ID NO: 113 - DNA (cDNA), 2347 bp; single-stranded, linear
aaaacacgggggaaatgacagtagcaaagaatctggactatgaagaatgctcattgtatgaaatggaaatacaggctgaagatgtgggggcgcttctggggaggagcaaagtggtaattatggtagaagatgtaaatgacaatcggccagaagtgaccattacatccttgtttaacccggtattggaaaattctcttcccgggacagtaattgccttcttgaatgtgcatgaccgagactctggaaagaacggccaagttgtctgttacacgcatgataacttaccttttaaattagaaaagtcaatagataattattatagattggtgacatggaaatatttggaccgagaaaaagtctccatctacaatatcacagtgatagcctcagatctaggagcccactctgtcactgaaacttacattgccctgattgtggcagacactaatgacaaccctcctcgttttcctcacacctcctacacagcctatattccagagaacaacctgaggggcgcctccatcttctcactgactgcacatgatcctgacagtcaggaaaatgcacaggtcacttactctgtgtctgaggacaccatacagggagtgcctttgtcctcttatatctccatcaactcagatactggtgtcctgtatgcactgcactcttttgacttcgagaagatacaagacttgcagctactggttgttgccactgacagtggaagcccacctctcagcagcaatgtgtcattgagcttgtttgtgttggaccagaacgacaacgcacctgagattctatatcctagcttccccacagatggctccactggtgtggaactagcaccccgctctgcagagcctggatacctagtgaccaaagtggtggcagtggacaaagactcaggacagaatgcttggctgtcctaccgtctgctgaaggccagcgaacctgggctcttctctgtaggacttcacacgggtgaggtgcgtacagcgagggccctgctggacagagatgctctcaaacagaatctggtgatggccgtgcaggaccatggccaaccccctctctcggccactgtaactctcactgtggcagtggctaacagcatccctgaggtgttggctgacttgagcagcattaggacccctggggtaccagaggattctgatatcacgctccacctggtggtggcagtggctgtggtctcctgtgtcttccttgtctttgtcattgtcctcctagctctcaggcttcagcgctggcagaagtctcgccagctccagggctccaaaggtggattggctcctgcacctccatcacattttgtgggcatcgacggggtacaggcttttctacaaacctattctcatgaagtctcgctcacttcaggctcccagacaagccacattatctttcctcagcccaactatgcagacatgctcattaaccaagaaggctgtgagaaaaatgattccttattaacatccatagattttcatgagagtaaccgtgaagatgcttgcgccccgcaagccccgcccaacactgactggcgtttctctcaagcccagagacccggcacgagcggatcccaaaatggggatgaaaccggcacctggcccaacaaccagttcgatacagagatgctgcaagccatgatcttggcctctgccagtgaagccgctgatgggagctccactctgggagggggcactggcactatgggtttgagcgctcgatatggaccccagtttaccctgcagcacgtgcctgactaccgccagaacgtgtacatccctggcagcaatgccacactgaccaacgcagctggcaaacgagatggcaaggctccggcaggcggcaatggcaacaacaacaagtcgggcaagaaagagaagaagtaatatggaggccaggccttgagccacagggcagcctccctccccagccagtccagcttgtccttacttgtacccaggcctcagaatttcagggctcaccccaggattctggtaggagccacagccaggccatgctccccgttgggaaacagaaacaagtgcccaagccaacaccccctctttgtaccctaggggggttgaatatgcaaagagagttctgctgggaccccctatccaatcagtgattgtacccacataggtagcagggttagtgtggatacacacacacacacacacacacacacacacacaacccttgtcctccgcagtgcctgccactttctgggactttctcatccccctacgcccttcctttatcctctcccacccagacacagctgctggagaataaatttggggatgctgatgctaaaaaaaaa

SEQ ID NO: 114 - DNA (cDNA), 2972 bp; single-stranded, linear
  CDS 2..1849
agaggctgctcaccacctggtcctcacggcctcggatggcggcaagccgcctcgctctagcacagtgcgcatccacgtgacagtgttggatacaaatgacaatgccccggtttttcctcacccgatttaccgagtgaaagtccttgagaacatgcccccaggcacgcggctgcttactgtaacagccagcgacccggatgagggaatcaacggaaaagtggcatacaaattccggaaaattaatgaaaaacaaactccgttattccagcttaatgaaaatactggggaaatatcaatagcaaaaagtctagattatgaagaatgttcattttatgaaatggaaatacaagccgaagatgtgggggcacttctggggaggaccaaattgctcatttctgtggaagatgtaaatgacaatagaccagaagtgatcattacgtctttgtttagcccagtgttagaaaattctcttcccgggacagtaattgccttcttgagtgtgcatgaccaagactctggaaagaatggtcaagttgtctgttacacacgtgataatttaccttttaaattagaaaagtcaataggtaattattatagattagtgacaaggaaatatttggaccgagaaaatgtctctatctacaatatcacagtgatggcctcagatctaggaacaccacctctgtccactgaaactcaaatcgctctgcacgtggcagacattaacgacaaccctcctactttccctcatgcctcctactcagcgtatatcctagagaacaacctgagaggagcctccatcttttccttgactgcacacgaccccgacagccaggagaatgcccaggtcacttactctgtgaccgaggacacgctgcagggggcgcccctgtcctcgtatatctccatcaactctgacaccggtgtcctgtatgcgctgcaatctttcgactatgagcagatccgagacctgcagctactggtaacagccagcgacagcggggacccgcccctcagcagcaacatgtcactgagcctgttcgtgctggaccagaatgacaacgcgcccgagatcctgtaccccgccctccccacagacggttccactggcgtggagctggcgccccgctccgcagagcgtggctacctggtgaccaaggtggtggcggtggacagagactcgggccagaacgcctggctgtcctaccgcctgctcaaggccagcgagccgggactcttctcggtgggtctgcacacgggcgaggtgcgcacggcgcgagccctgctggacagagacgcgctcaagcagagcctcgtggtggccgtccaggaccatggccagccccctctctccgccactgtcacgctcaccgtagccgtggctgacagcatccccgaagtcctgaccgagttgggcagtctgaagccttcggtcgacccgaacgattcgagccttacactctatctcgtggtggcagtggctgccatctcctgtgtcttcctcgcctttgtcgctgtgcttctggggctcaggctgaggcgctggcacaagtcacgcctgctccaggattccggtggcagattggtaggcgtgcctgcctcacattttgtgggtgttgaggaggtacaggctttcctgcagacctattcccaggaagtctccctcaccgccgactcgcggaagagtcacctgatctttccccagcccaactacgcagacatgctcatcagtcaggagggctgtgagaaaaatgattctttgttaacatccgtagattttcatgaatataagaatgaagctgatcatggtcaggtgagtttagttctttgcttgcttttaatttccagatgaattttatttggcataaattatgttttgaaaaacattgtgaagatagttgaaaataatttttaaggtgtatcacagagttttgggtttattttggtggtgttaccaaaaaattgaactctaatagtcataggttattgtttcatttgcttttaaacgacttggaaaagattgttccaccattttaaaccttccagtattttattcctattatcactcattcacttaagaagtagctacccgtccatactggtaattttgctattgtttgtttgtgtgtgtgtgtgtgtgtgtgtgtgtgtgtgtgtatcccaaactagaacttcagaaaattatcaagaagtctaaagccttgttattagcttagcaaaagtaaaatatatctcagaatttttagggttatgtttagcatttgaacctgtaactaggctcttgtatatttcttcactttaaacctcttttctgagccctgtttctgtaccagtgcccttcaaaactttaatacttcttaccatccttcaaaacatgaacaaactttaaagatggatcttggtgggagatgagactggttactaaatattaagtatgtgagtcagtggtcacctgggctccatccccatggagacatgaaatctaaagcctagaatgtccattgctcccccaaacaaaaaacaaaagcaaaaacattagatctgaattaaaatgtaattttaaactgttgaaagtgacttttgtaaaatatgtaagaacatatttcaatacaattccaattagctgtttcggttgtgcattgatgtgaagtggtgagaatgttgatattaagaaccaatgtttcaggtacacaagttctaaataagctgatcaattcaattaaagttattcagtcttggctggacacagtgcctcatgtctgaaatcccagcactttgggaggctggggcaggaggaccgcttgagccccgggggtttgaaactgcagtgagctatgatcatgccactgcactccagcctaggtggcagaactagaccctgtctctaaaaaaactattattaggccgcgtgcggtggctcacgcctgtaatcccagcactttgggagactgaggtgggtggatcacctgagc

SEQ ID NO: 115 - protein, 616 aa
EAAHHLVLTASDGGKPPRSSTVRIHVTVLDTNDNAPVFPHPIYRVKVLENMPPGTRLLTVTASDPDEGINGKVAYKFRKINEKQTPLFQLNENTGEISIAKSLDYEECSFYEMEIQAEDVGALLGRTKLLISVEDVNDNRPEVIITSLFSPVLENSLPGTVIAFLSVHDQDSGKNGQVVCYTRDNLPFKLEKSIGNYYRLVTRKYLDRENVSIYNITVMASDLGTPPLSTETQIALHVADINDNPPTFPHASYSAYILENNLRGASIFSLTAHDPDSQENAQVTYSVTEDTLQGAPLSSYISINSDTGVLYALQSFDYEQIRDLQLLVTASDSGDPPLSSNMSLSLFVLDQNDNAPEILYPALPTDGSTGVELAPRSAERGYLVTKVVAVDRDSGQNAWLSYRLLKASEPGLFSVGLHTGEVRTARALLDRDALKQSLVVAVQDHGQPPLSATVTLTVAVADSIPEVLTELGSLKPSVDPNDSSLTLYLVVAVAAISCVFLAFVAVLLGLRLRRWHKSRLLQDSGGRLVGVPASHFVGVEEVQAFLQTYSQEVSLTADSRKSHLIFPQPNYADMLISQEGCEKNDSLLTSVDFHEYKNEADHGQVSLVLCLLLISR

(19) United States
(12) Patent Application Publication
(10) Pub. No.: US 2006/0024291 A1
(43) Pub. Date: Feb. 2, 2006

(54) Protocadherin materials and methods

(76) Inventor: Shintaro Suzuki, Torrance, CA (US)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 11/154,227

(22) Filed: Jun. 16, 2005

Related U.S. Application Data
(63) Continuation of application No. 09/880,573, filed on Jun. 13, 2001.
Application No. 09/880,573 is a continuation-in-part of application No. PCT/US93/12588, filed on Dec. 23, 1993.
Application No. PCT/US93/12588 is a continuation-in-part of application No. 07/998,003, filed on Dec. 29, 1992, now Pat. No. 5,643,781.

(51) Int. Cl. C07H 21/04; C12P 21/06; A61K 39/395; C07K 14/705; C07K 16/28

(52) U.S. Cl. 424/130.1; 530/387.1; 530/350; 435/69.1; 435/320.1; 435/325; 536/23.5

(57) ABSTRACT

Polynucleotide sequences encoding novel cadherin-like polypeptides, designated protocadherins, and variants thereof are provided by the invention as well as methods and materials for the recombinant production of the same. Antibody substances specific for protocadherins are also disclosed as useful for modulating the natural binding and/or regulatory activities of the protocadherins.

This application is a continuation-in-part of International Patent Application No. PCT/US93/12588 filed Dec. 23, 1993 which is in turn a continuation-in-part of U.S. patent application Ser. No. 07/998,003 which was filed on Dec. 29, 1992.

### FIELD OF THE INVENTION

The present invention relates, in general, to materials and methods relevant to cell-cell adhesion. More particularly, the invention relates to novel adhesion proteins, designated protocadherins, and to polynucleotide sequences encoding the protocadherins. The invention also relates to methods for inhibiting binding of the protocadherins to their natural ligands/antiligands.

### BACKGROUND

In vivo, intercellular adhesion plays an important role in a wide range of events including morphogenesis and organ formation, leukocyte extravasion, tumor metastasis and invasion, and the formation of cell junctions. Additionally, cell-cell adhesion is crucial for the maintenance of tissue integrity.

Intercellular adhesion is mediated by specific cell surface adhesion molecules. Cell adhesion molecules have been classified into at least four families including the immunoglobulin superfamily, the integrin superfamily, the selectin family and the cadherin superfamily. All cell types that form solid tissues express some members of the cadherin superfamily suggesting that cadherins are involved in selective adhesion of most cell types.

Cadherins have been generally described as glycosylated integral membrane proteins that have an N-terminal extracellular domain (the N-terminal 113 amino acids of the domain appear to be directly involved in binding) consisting of five subdomains characterized by sequences unique to cadherins, a hydrophobic membrane-spanning domain and a C-terminal cytoplasmic domain that interacts with the cytoskeleton through catenins and other cytoskeleton-associated proteins. Some cadherins lack a cytoplasmic domain, however, and appear to function in cell-cell adhesion by a different mechanism than cadherins having a cytoplasmic domain. The cytoplasmic domain is required for the adhesive function of the extracellular domain in cadherins that do have an cytoplasmic domain. Binding between members of the cadherin family expressed on different cells is homophilic (i.e., a member of the cadherin family binds to cadherins of its own or a closely related subclass) and Ca²⁺-dependent. For recent reviews on cadherins, see Takeichi, Annu. Rev. Biochem., 59: 237-252 (1990) and Takeichi, Science, 251: 1451-1455 (1991).

The first cadherins to be described (E-cadherin in mouse epithelial cells, L-CAM in avian liver, uvomorulin in the mouse blastocyst, and CAM 120/80 in human epithelial cells) were identified by their involvement in Ca2+-dependent cell adhesion and their unique immunological characteristics and tissue localization. With the later immunological identification of N-cadherin, which was found to have a different tissue distribution than E-cadherin, it became apparent that a new family of Ca²⁺-dependent cell-cell adhesion molecules had been discovered.

The molecular cloning of the genes encoding E-cadherin [see Nagafuchi et al., Nature, 329: 341-343 (1987)], N-cadherin [Hatta et al., J. Cell. Biol., 106: 873-881 (1988)], and P-cadherin (Nose et al., EMBO J., 6: 3655-3661 (1987)] provided structural evidence that the cadherins comprised a family of cell adhesion molecules. Cloning of L-CAM [Gallin et al., Proc. Natl. Acad. Sci. USA, 84: 2808-2812 (1987)] and uvomorulin [Ringwald et al., EMBO J., 6: 3647-3653 (1986)] revealed that they were identical to E-cadherin. Comparisons of the amino acid sequences of E-, N-, and P-cadherins showed a level of amino acid similarity of about 45%-58% among the three subclasses. Liaw et al., EMBO J., 9: 2701-2708 (1990) describes the use of PCR with degenerate oligonucleotides based on conserved regions of the E-, N- and P-cadherins to amplify N- and P-cadherin from a bovine microvascular endothelial cell cDNA.

The isolation by PCR of eight additional cadherins was reported in Suzuki et al., Cell Regulation, 2: 261-270 (1991). Subsequently, several other cadherins were described including R-cadherin [Inuzuka et al., Neuron, 7: 69-79 (1991)], M-cadherin [Donalies, Proc. Natl. Acad. Sci. USA, 88: 8024-8028 (1991)], B-cadherin [Napolitano, J. Cell. Biol., 113: 893-905 (1991)] and T-cadherin Ranscht, Neuron, 7: 391-402 (1991)].

Additionally, proteins distantly related to cadherins such as desmoglein [Goodwin et al., Biochem. Biophys. Res. Commun., 173: 1224-1230 (1990) and Koch et al., Eur. J. Cell Biol., 53: 1-12 (1990)] and the desmocollins [Holton et al., J. Cell Science, 97: 239-246 (1990)] have been described. The extracellular domains of these molecules are structurally related to the extracellular domains of typical cadherins, but each has a unique cytoplasmic domain. Mahoney et al., Cell, 67: 853-868 (1991) describes a tumor suppressor gene of Drosophila, called fat, that also encodes a cadherin-related protein. The fat tumor suppressor comprises 34 cadherin-like subdomains followed by four EGF-like repeats, a transmembrane domain, and a novel cytoplasmic domain. The identification of these cadherin-related proteins is evidence that a large superfamily characterized by a cadherin extracellular domain motif exists.

Studies of the tissue expression of the various cadherin-related proteins reveal that each subclass of molecule has a unique tissue distribution pattern. For example, E-cadherin is found in epithelial cells while N-cadherin is found in neural and muscle cells. Expression of cadherin-related proteins also appears to be spatially and temporally regulated during development because individual proteins appear to be expressed by specific cells and tissues at specific developmental stages [for review see Takeichi (1991), supra]. Both the ectopic expression of cadherin-related proteins and the inhibition of native expression of cadherin-related proteins hinders the formation of normal tissue structure [Detrick et al., Neuron, 4: 493-506 (1990); Fujimori et al., Development, 110: 97-104 (1990); Kintner, Cell, 69: 225-236 (1992)].

The unique temporal and tissue expression pattern of the different cadherins and cadherin-related proteins is particularly significant when the role each subclass of proteins may play in vivo in normal events (e.g., the maintenance of the intestinal epithelial barrier) and in abnormal events (e.g., tumor metastasis or inflammation) is considered. Different subclasses or combinations of subclasses of cadherin-related proteins are likely to be responsible for different cell-cell adhesion events in which therapeutic detection and/or intervention may be desirable. For example, auto-antibodies from patients with pemphigus vulgaris, an autoimmune skin disease characterized by blister formation caused by loss of cell adhesion, react with a cadherin-related protein offering direct support for adhesion function of cadherins in vivo [Amagai et al., Cell, 67: 869-877 (1991)]. Studies have also suggested that cadherins and cadherin-related proteins may have regulatory functions in addition to adhesive activity. Matsunaga et al., Nature, 334: 62-64 (1988) reports that N-cadherin has neurite outgrowth promoting activity. The Drosophila fat tumor supressor gene appears to regulate cell growth and supress tumor invasion as does mammalian E-cadherin [see Mahoney et al., supra; Frixen et al., J. Cell. Biol., 113:173-185 (1991); Chen et al., J. Cell, Biol., 114:319-327 (1991); and Vleminckx et al., Cell, 66:107-119 (1991)]. Thus, therapeutic intervention in the regulatory activities of cadherin-related proteins expressed in specific tissues may be desirable.

There thus continues to exist a need in the art for the identification and characterization of additional cadherin-related proteins which participate in cell-cell adhesion and/or regulatory events. Moreover, to the extent that cadherin-related proteins might form the basis for the development of therapeutic and diagnostic agents, it is essential that the genes encoding the proteins be cloned. Information about the DNA sequences and amino acid sequences encoding the cadherin-related proteins would provide for the large scale production of the proteins by recombinant techniques and for the identification of the tissues/cells naturally producing the proteins. Such sequence information would also permit the preparation of antibody substances or other novel binding molecules specifically reactive with the cadherin-related proteins that may be useful in modulating the natural ligand/antiligand binding reactions in which the proteins are involved.

### SUMMARY OF THE INVENTION

The present invention provides cadherin-related materials and methods that are relevant to cell-cell adhesion. In one of its aspects, the present invention provides purified and isolated polynucleotides (e.g., DNA and RNA, both sense and antisense strands) encoding the novel cell adhesion molecules designated herein as protocadherins, including protocadherin-42, protocadherin-43, protocadherin pc3, protocadherin pc4 and protocadherin pc5. Preferred polynucleotide sequences of the invention include genomic and cDNA sequences as well as wholly or partially synthesized DNA sequences, and biological replicas thereof (i.e., copies of the sequences made in vitro). Biologically active vectors comprising the polynucleotide sequences are also contemplated.

Specifically illustrating protocadherin polynucleotide sequences of the present invention are the inserts in the plasmids pRC/RSV-pc42 and pRC/RSV-pc43 which were deposited with the American Type Culture Collection (ATCC), 12301 Parklawn Drive, Rockville, Md. 20852 on Dec. 16, 1992 and were assigned ATCC Accession Nos. 69162 and 69163, respectively.

The scientific value of the information contributed through the disclosures of the DNA and amino acid sequences of the present invention is manifest. For example, knowledge of the sequence of a partial or complete DNA encoding a protocadherin makes possible the isolation by standard DNA/DNA hybridization or PCR techniques of full length cDNA or genomic DNA sequences that encode the protein (or variants thereof) and, in the case of genomic DNA sequences, that specify protocadherin-specific regulatory sequences such as promoters, enhancers and the like. Alternatively, DNA sequences of the present invention may be chemically synthesized by conventional techniques.

Hybridization and PCR techiques also allow the isolation of DNAs encoding heterologous species proteins homologous to the protocadherins specifically illustrated herein.

According to another aspect of the invention, host cells, especially eucaryotic and procaryotic cells, are stably transformed or transfected with the polynucleotide sequences of the invention in a manner allowing the expression of protocadherin polypeptides in the cells. Host cells expressing protocadherin polypeptide products, when grown in a suitable culture medium, are particularly useful for the large scale production of protocadherin polypeptides, fragments and variants thereby enabling the isolation of the desired polypeptide products from the cells or from the medium in which the cells are grown.

The novel protocadherin protein products of the invention may be obtained as isolates from natural tissue sources, but are preferably produced by recombinant procedures involving the host cells of the invention. The products may be obtained in fully or partially glycosylated, partially or wholly de-glycosylated, or non-glycosylated forms depending on the host cell selected or recombinant production and/or post-isolation processing.

Protocadherin variants according to the invention may comprise polypeptide analogs wherein one or more of the specified amino acids is deleted or replaced or wherein one or more non-naturally encoded amino acids are added: (1) without loss, and preferably with enhancement, of one or more of the biological activities or immunological characteristics specific for a protocadherin; or (2) with specific disablement of a particular ligand/antiligand binding function. Also contemplated by the present invention are antibody substances (e.g., monoclonal and polyclonal antibodies, chimeric and humanized antibodies, antibody domains including Fab, Fab′, F(ab′)₂, Fv or single variable domains, and single chain antibodies) which are specific for the protocadherins of the invention. Antibody substances can be developed using isolated natural, recombinant or synthetic protocadherin polypeptide products or host cells expressing such products on their surfaces. The antibody substances may be utilized for purifying protocadherin polypeptides of the invention, for determining tissue expression of polypeptides and as antagonists of the ligand/antiligand binding activities of the protocadherins. Specifically illustrating monoclonal antibodies of the present invention are the protocadherin-43 specific monoclonal antibodies produced by the hybridoma cell line designated 3812C which was deposited with the ATCC on Dec. 2, 1992 and was assigned ATCC Accession No. HB 11207.

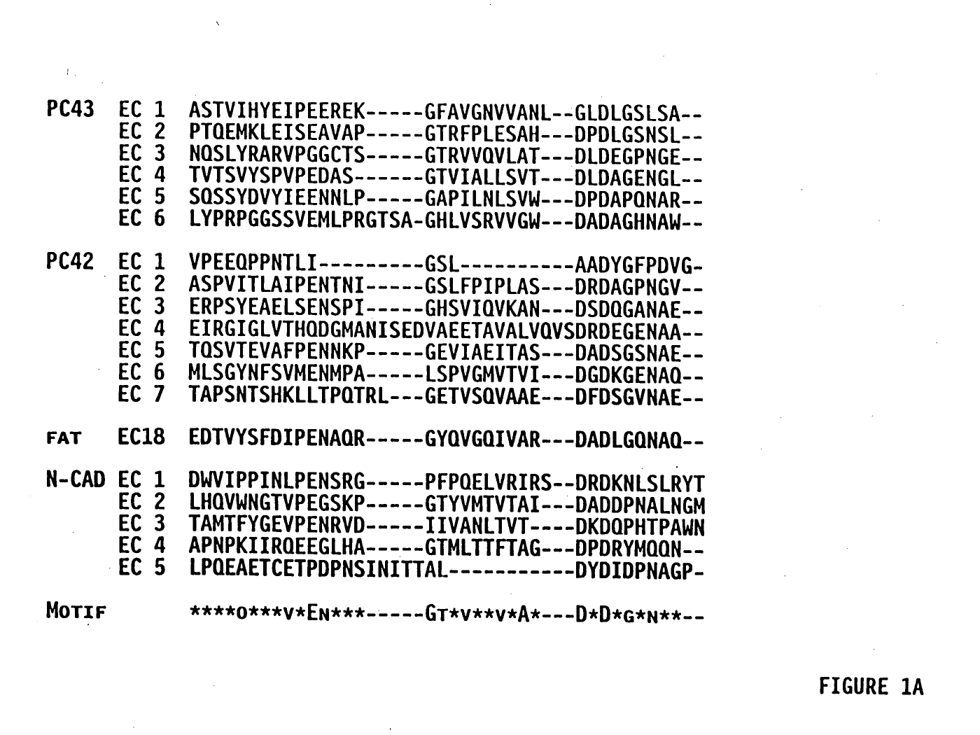

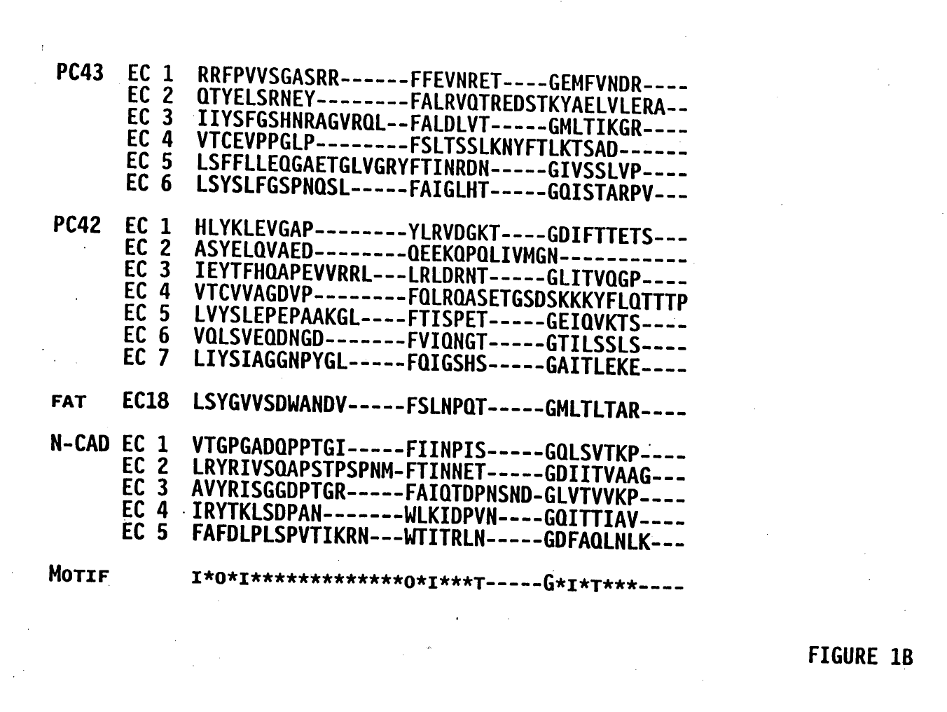

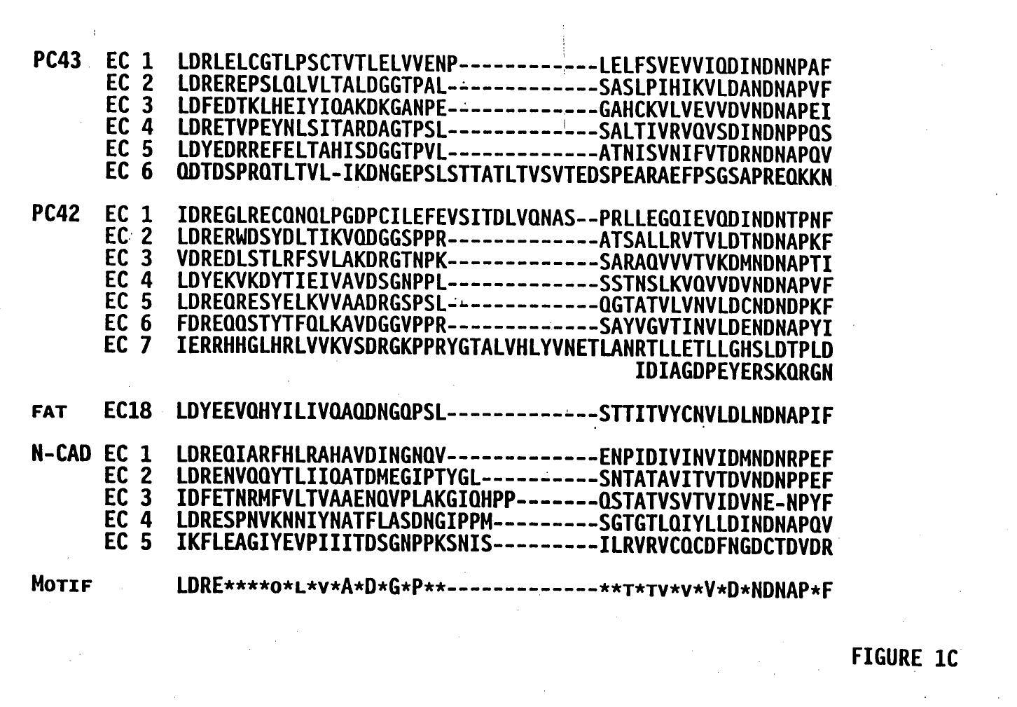

Numerous other aspects and advantages of the present invention will be apparent upon consideration of the following detailed description, reference being made to the drawing wherein FIG. 1A-C is an alignment of protocadherin amino acid sequences of the invention with the amino acid sequences of N-cadherin and of the Drosophila fat tumor suppressor.

### DETAILED DESCRIPTION

The present invention is illustrated by the following examples wherein Examples 1, 2 and 3 describe the isolation by PCR of protocadherin polynucleotide sequences. Example 3 also describes the chromosome localization of several protocadherin genes of the invention. Example 4 describes the isolation by DNA/DNA hybridization of additional protocadherin polynucleotide sequences of the present invention. Example 5 presents the construction of expression plasmids including polynucleotides encoding protocadherin-42 or protocadherin-43 and the transfection of L cells with the plasmids. The generation of antibodies to protocadherin-42 and protocadherin-43 is described in Example 6. Example 7 presents the results of immunoassays of transfected L cells for the expression of protocadherin-42 or protocadherin-43. Example 8 describes the cell aggregation properties of L cells transfected with protocadherin-42, protocadherin-43 or a chimeric protocadherin-43/E-cadherin molecule. The calcium-binding properties of pc43 are described in Example 9. The results of assays of various tissues and cell lines for the expression of protocadherin-42 and protocadherin-43 by Northern blot, Western blot and in situ hybridization are respectively presented in Examples 10, 11 and 12. Example 13 describes immunoprecipitation experiments identifying a 120 kDa protein that coprecipitates with protocadherin-43.

### EXAMPLE 1

The polymerase chain reaction (PCR) was used to isolate novel rat cDNA fragments encoding cadherin-related polypeptides.

Design of PCR Primers

Two regions of conserved amino acid sequence, one from the middle of the third cadherin extracellular subdomain (EC-3) and the other from the C-terminus of the fourth extracellular subdomain (EC-4), were identified by comparison of the published amino acid sequences for L-CAM (Gallin et al., supra), E-cadherin (Nagafuchi et al., supra), mouse P-cadherin (Nose et al., supra), uvomorulin (Ringwald et al., supra), chicken N-cadherin (Hatta et al., supra), mouse N-cadherin [Miyatani et al., Science, 245:631-635 (1989)] and human P-cadherin [Shimoyama et al., J. Cell. Biol., 109:1787-1794 (1989)], and the corresponding degenerate oligonucleotides respectively set out below in IUPAC-IUB Biochemical nomenclature were designed for use as PCR primers.

Primer 1

(SEQ ID NO: 1)

5′ AARSSNNTNGAYTRYGA 3′
Primer 2

(SEQ ID NO: 2)

3′ TTRCTRTTRCGNGGNNN 5′

The degenerate oligonucleotides were synthesized using an Applied Biosystems model 380B DNA synthesizer (Foster City, Calif.).
Cloning of cDNA Sequences by PCR

PCR was carried out in a manner similar to that described in Suzuki et al., Cell Regulation, 2: 261-270 (1991) on a rat brain cDNA preparation. Total RNA was prepared from rat brain by the guanidium isothiocyanate/cesium chloride method described in Maniatis et al., pp. 196 in Molecular Cloning: A Laboratory Manual, Cold Spring Harbor, N.Y.: Cold Spring Harbor Laboratory (1982). Brain poly(A)⁺ RNAs were then isolated using a FastTrack® kit (Invitrogen, San Diego, Calif.) and cDNA was prepared using a cDNA synthesis kit (Boehringer Mannheim Biochemicals, Indianapolis, Ind.). The PCR reaction was initiated by adding 2.5 units of Taq DNA polymerase (Boehringer Mannheim Biochemicals) to 100 ng template cDNA and 10 μg of each primer, after which 35 reaction cycles of denaturation at 94° C. for 1.5 minutes, annealing at 45° C. for 2 minutes, and polymerization at 72° C. for 3 minutes were carried out. Two major bands of about 450 base pairs (bp) and 130 bp in size were found when the products of the PCR reaction were subjected to agarose gel electrophoresis. The 450 bp band corresponded to the expected length between the two primer sites corresponding to the middle of the third cadherin extracellular subdomain (EC-3) and the carboxyl terminus of the fourth cadherin extracellular subdomain (EC-4), but the 130 bp band could not be predicted from any of the previously identified cadherin sequences. The 450 bp and 130 bp bands were extracted by a freezing and thawing method. The resulting fragments were phosphorylated at the 5′ end with T4 polynucleotide kinase and subcloned by a blunt-end ligation into the Sma I site of M13mp18 (Boehringer Mannheim Biochemicals) in a blunt end ligation for sequence analysis. Sequencing of the fragments was carried out by the dideoxynucleotide chain termination method using a Sequenase kit (United States Biochemicals, Cleveland, Ohio). DNA and amino acid sequence were analyzed using the Beckman Microgenie program (Fullerton, Calif.).

Analysis of cDNA Sequences

Nineteen novel partial cDNA clones were isolated. The DNA and deduced amino acid sequences of the clones (including sequences corresponding to the PCR primers) are set out as follows: RAT-123 (SEQ ID NOs: 3 and 4, respectively), RAT-212 (SEQ ID NOs: 5 and 6), RAT-214 (SEQ ID NOs: 7 and 8), RAT-216 (SEQ ID NOs: 9 and 10), RAT-218 (SEQ ID NOs: 11 and 12), RAT-224 (SEQ ID NOs: 13 and 14), RAT-312 (SEQ ID NOs: 15 and 16), RAT-313 (SEQ ID NOs: 17 and 18), RAT-314 (SEQ ID NOs: 19 and 20), RAT-315 (SEQ ID NOs: 21 and 22), RAT-316 (SEQ ID NOs: 23 and 24), RAT-317 (SEQ ID NOs: 25 and 26), RAT-321 (SEQ ID NOs: 27 and 28), RAT-323 (SEQ ID NOs: 29 and 30), RAT-336 (SEQ ID NOs: 31 and 32), RAT-352 (SEQ ID NOs: 33 and 34), RAT-411 (SEQ ID NOs: 35 and 36), RAT-413 (SEQ ID NOs: 37 and 38), and RAT-551 (SEQ ID NOs: 39 and 40).

The deduced amino acid sequences of the cDNA clones are homologous to, but distinct from the known cadherins. The cadherins described thus far have highly conserved, short amino acid sequences in the third extracellular subdomain (EC-3) including the consensus sequence D-Y-E or D-F-E located at the middle region of the subdomain and the consensus sequence D-X-N-E-X-P-X-F (SEQ ID NO: 41) or D-X-D-E-X-P-X-F (SEQ ID NO: 42) at its end (Hatta et al., supra), while the corresponding sequences of other subdomains, except for the fifth extracellular subdomain (EC-5), are D-R-E and D-X-N-D-N-X-P-X-F (SEQ ID NO: 43), respectively. In contrast, the deduced amino acid sequences of the new clones that correspond to cadherin extracellular subdomains include the sequence D-Y-E or D-F-E at one end, but have the sequence D-X-N-D-N-X-P-X-F instead of D-X-N-E-X-P-X-F or D-X-D-E-X-P-X-F, at the other end. The polypeptides encoded by the partial clones are homologous to previously identified cadherins but did not show significant homology to any other sequences in Genbank. Therefore, the partial cDNAs appear to comprise a new subclass of cadherin-related molecules.

### EXAMPLE 2

Various cDNA fragments structurally similar to the rat cDNAs described in Example 1 were isolated from human, mouse, and Xenopus brain cDNA preparations and from Drosophila and C. elegans whole body cDNA preparations by PCR using Primers 1 and 2 as described in Example 1. The DNA and deduced amino acid sequences of the resulting PCR fragments (including sequences corresponding to the PCR primers) are set out as follows: MOUSE-321 (SEQ ID NOs: 44 and 45), MOUSE-322 (SEQ ID NOs: 46 and 47), MOUSE-324 (SEQ ID NOs: 48 and 49), MOUSE-326 (SEQ ID NOs: 50 and 51), HUMAN-11 (SEQ ID NOs: 52 and 53), HUMAN-13 (SEQ ID NOs: 54 and 55), HUMAN-21 (SEQ ID NOs: 56 and 57), HUMAN-24 (SEQ ID NOs: 58 and 59), HUMAN-32 (SEQ ID NOs: 60 and 61), HUMAN-42 (SEQ ID NOs: 62 and 63), HUMAN-43 (SEQ ID NOs: 64 and 65), HUMAN-212 (SEQ ID NOs: 66 and 67), HUMAN-213 (SEQ ID NOs: 68 and 69), HUMAN-215 (SEQ ID NOs: 70 and 71), HUMAN-223 (SEQ ID NOs: 72 and 73), HUMAN-410 (SEQ ID NOs: 74 and 75), HUMAN-443 (SEQ ID NOs: 76 and 77), XENOPUS-21 (SEQ ID NOs: 78 and 79), XENOPUS-23 (SEQ ID NOs: 80 and 81), XENOPUS-25 (SEQ ID NOs: 82 and 83), XENOPUS-31 (SEQ ID NOs: 84 and 85), DROSOPHILA-12 (SEQ ID NOs: 86 and 87), DROSOPHILA-13 (SEQ ID NOs: 88 and 89), DROSOPHILA-14 (SEQ ID NOs: 90 and 91) and C.ELEGANS-41 (SEQ ID NOs: 92 and 93). Comparison of the deduced amino acid sequences indicates significant similarity between sets of these clones. In particular, there are three sets of clones that appear to be cross-species homologues: RAT-218, MOUSE-322 and HUMAN-43; RAT-314, MOUSE-321 and HUMAN-11; and MOUSE-326 and HUMAN-42.

### EXAMPLE 3

To ascertain the complete structure of the new proteins defined by the PCR products, two full length human cDNAs corresponding to the partial cDNAs HUMAN-42 and HUMAN-43 were isolated.

Isolation of Full-Length Human cDNAs

A human fetal brain cDNA library (Stratagene, La Jolla, Calif.) in the λZapII vector was screened by the plaque hybridization method [described in Ausubel et al., Eds., Current Protocols in Molecular Biology, Sections 6.1.1 to 6.1.4 and 6.2.1 to 6.2.3, John Wiley & Sons, New York (1987)] with ³²P-labelled HUMAN-42 and HUMAN-43 DNA fragments. The positive clones were plaque-purified and, using a helper virus, the inserts were cut out by an in vivo excision method in the form of a Bluescript SK(+) plasmid. The insert sequences were then subcloned into the M13 vector (Boehringer Mannheim, Biochemicals) for sequencing. Several overlapping cDNA clones were isolated with each probe including two cDNAs which contained the putative entire coding sequences of two novel proteins designated protocadherin-42 (pc42) and protocadherin-43 (pc43). The DNA and deduced amino acid sequences of pc42 are set out in SEQ ID NOs: 94 and 95, respectively, while the DNA and deduced amino acid sequences of pc43 are set out in SEQ ID NOs: 96 and 97, respectively.

A description of the cloning of protocadherin sequences of the invention was published in Sano et al., The EMBO Journal, 12(6): 2249-2256 (1993) after filing of the priority application hereto. The deduced amino acid sequence of pc43 was previously presented at the Dec. 9, 1991 meeting of the American Society for Cell Biology. An abstract of the presentation is published as Suzuki et al., J. Cell. Biol., 115: 72a (Abstract 416) (Dec. 9, 1991).

Analysis of Full-Length Human Clones

Comparison of the full length cDNA sequences of pc42 and pc43 to the sequences of the various DNA fragments originally obtained by PCR reveals that MOUSE-326 and HUMAN-42 correspond to a portion of the fourth extracellular subdomain (EC-4) of pc42, and RAT-314, MOUSE-321, and HUMAN-11 correspond to a portion of the third extracellular subdomain (EC-3) of pc43 and RAT-218, MOUSE-322 and HUMAN-43 correspond to a portion of the fifth extracellular domain (EC-5) of pc43.

The overall structures of pc42 and pc43 are similar to that of typical cadherins but the new molecules also have distinct features. Both protocadherin cDNA sequences contain putative translation initiation sites and translated amino acid sequences start with typical signal sequences, but the clones lack the prosequences that are present in all known cadherin precursors. The cDNAs encode proteins having a large N-terminal extracellular domain and a relatively short C-terminal cytoplasmic domain connected by a transmembrane sequence. The extracellular domains of pc42 and pc43 are different in length and pc42 contains seven subdomains that closely resemble the typical cadherin extracellular subdomain while pc43 has six such subdomains. The sizes of the protocadherin cytoplasmic domains are similar to those of typical cadherins, but the sequences do not show any significant homology with those of known cadherins or cadherin-related proteins.

Amino acid identity determinations between extracellular subdomains of human pc42 and pc43, and of mouse N-cadherin (SEQ ID NO: 98) (presented as an example of a “typical” cadherin) and the eighteenth extracellular subdomain of Drosophila fat tumor suppressor (EC-18, SEQ ID NO: 99) (the eighteenth extracellular subdomain of fat is a prototypical fat subdomain) are presented in Table 1 below, wherein, for example, “N-EC-1×pc42indicates that the first extracellular subdomain of N-cadherin was compared to the extracellular subdomain of pc42 indicated on the horizonal axis.

TABLE 1

| | EC-1 | EC-2 | EC-3 | EC-4 | EC-5 | EC-6 | EC-7 |
|---|---|---|---|---|---|---|---|
| N-EC-1 × pc42 | 20 | 27 | 26 | 26 | 31 | 29 | 17 |
| N-EC-1 × pc43 | 31 | 23 | 23 | 26 | 31 | 24 | |
| N-EC-2 × pc42 | 28 | 30 | 32 | 30 | 37 | 31 | 19 |
| N-EC-2 × pc43 | 30 | 28 | 30 | 36 | 29 | 30 | |
| N-EC-3 × pc42 | 21 | 26 | 30 | 29 | 31 | 30 | 22 |
| N-EC-3 × pc43 | 25 | 18 | 26 | 28 | 28 | 25 | |
| N-EC-4 × pc42 | 28 | 28 | 26 | 25 | 29 | 27 | 17 |
| N-EC-4 × pc43 | 21 | 25 | 28 | 28 | 29 | 24 | |
| N-EC-5 × pc42 | 24 | 21 | 25 | 24 | 24 | 19 | 12 |
| N-EC-5 × pc43 | 15 | 21 | 20 | 20 | 25 | 16 | |
| fat EC-18 × pc42 | 22 | 35 | 32 | 34 | 42 | 35 | 19 |
| fat EC-18 × pc43 | 32 | 30 | 36 | 36 | 33 | 29 | |

The amino acid identity values between the extracellular subdomains of pc42 and pc43, and N-cadherin EC-1 through EC-5 and Drosophila fat EC-18 are mostly less than 40%. These identity values are comparable to the values between the subdomains of other cadherin subclasses. However, higher identity values indicate that pc42 and pc43 are more closely related to fat than to N-cadherin.

Amino acid identity determinations between extracellular subdomains of human pc42 and pc43 are presented in Table 2 below.

TABLE 2
pc42

| pc43 | EC-1 | EC-2 | EC-3 | EC-4 | EC-5 | EC-6 | EC-7 |
|---|---|---|---|---|---|---|---|
| EC-1 | 33 | 27 | 29 | 26 | 25 | 26 | 25 |
| EC-2 | 26 | 38 | 29 | 33 | 34 | 28 | 21 |
| EC-3 | 26 | 32 | 41 | 30 | 32 | 31 | 22 |
| EC-4 | 25 | 34 | 30 | 41 | 39 | 31 | 18 |
| EC-5 | 23 | 32 | 29 | 27 | 36 | 34 | 16 |
| EC-6 | 25 | 25 | 26 | 25 | 28 | 23 | 26 |

The identity values between respective EC-1, EC-2, EC-3, EC-4, EC-5 subdomains and the last subdomains of pc42 and pc43 are generally higher values than values obtained for comparisons of the protocadherins to N-cadherin. These results suggest that pc42 and pc43 are more closely related to one another than they are to classic cadherins.

FIG. 1A-C presents an alignment of the deduced amino acid sequences of the extracellular subdomains of pc42 (EC-1 through EC-7), pc43 (EC-1 through EC-6), mouse N-cadherin (EC-1 through EC-5) and Drosophila fat EC-18. A sequence on a line in FIG. 1A continues on the same line in FIGS. 1B and 1C. Gaps were introduced to maximize homology. The amino acid residues described by capital letters in the “motif” line are present in more than half of the subdomains of N-cadherin, pc42, pc43 and Drosophila fat. The amino acid residues described by small letters in the motif line are less well conserved in human pc42, pc43, and Drosophila fat. FIG. 1A-C shows that many amino acids characteristic of other cadherin extracellular domain repeats are conserved in the pc42 and pc43 sequences, including the cadherin sequence motifs DXD, DRE and DXNDNXPXF (SEQ ID NO: 43), two glycine residues, and one glutamic acid residue. Additionally, pc42 and pc43 share unique features in comparison to N-cadherin. More amino acids at specific sites are conserved between pc42 and pc43, such as the DXDXGXN (SEQ ID NO: 100) protocadherin sequence motif near the amino terminus of the pc42 and pc43 subdomains and the AXDXGXP (SEQ ID NO: 101) sequence motif near the carboxyl terminus of the subdomains. Additionally, both protocadherins share regions that do not show significant homology with the typical cadherin motif (of N-cadherin) near the carboxyl terminus of EC-1, in the middle of EC-2 and EC-4, and at the carboxyl terminus of the last repeat. A cysteine residue is located at a similar position in the middle of EC-4 of pc42 and pc43. In general, the extracellular subdomains of pc42 and pc43 are more similar to EC-18 of fat than the extracellular subdomains of N-cadherin.

Possible Alternative Splicing

Sequence analysis of various overlapping protocadherin cDNA clones revealed that some clones contained unique sequences at the 3′ end, although the 5′ end sequences were identical to other clones. The sequences forming the boundaries of the 3′ end regions are consistent with the consensus sequence of mRNA splicing, suggesting that these clones may correspond to alternatively spliced mRNAs. The DNA and deduced amino acid sequences of one possible product of alternative splicing of pc42 mRNA are set out in SEQ ID NOs: 102 and 103. The DNA and deduced amino acid sequences of two possible products of alternative splicing of pc43 mRNA are respectively presented in SEQ ID NO: 104 and 105, and SEQ ID NOs: 106 and 107.

Chromosome Localization

The chromosomal location of the protocadherin 413 gene (SEQ ID NO: 37) and of the pc42 and pc43 genes was determined by conventional methods.

Briefly, C3H/HeJ-gld and Mus spretus (Spain) mice and [(C3H/HeJ-gld×Mus spretus) F₁×C3H/HeJ-gld] interspecies backcross mice were bred and maintained as previously described in Seldin, et al., J. Exp. Med., 167: 688-693 (1988). Mus spretus was chosen as the second parent in the cross because of the relative ease of detection of informative restriction fragment length variants (RFLVs) in comparison with crosses using conventional inbred laboratory strains. Gene linkage was determined by segregation analysis.

Genomic DNA isolated from mouse organs by standard techniques was digested with restriction endonucleases and 10 μg samples were electrophoresed in 0.9% agarose gels. DNA was transferred to Nytran membranes (Schleicher & Schull, Inc., Keene, N.H.), hybridized with the appropriate probe at 65° C. and washed under stringent conditions, all as previously described in Maniatis et al., supra). To localize the pc42 gene, a mouse sequence probe corresponding to nucleotides 1419 to 1906 of SEQ ID NO: 94 was used and for pc43 a rat sequence probe corresponding to nucleotides 1060 to 1811 of SEQ ID NO: 96 was used. To localize the procadherin 413 gene, a probe including the sequence set out in SEQ ID NO: 37 was used. Other clones used as probes in the current study and RFLVs used to detect anonymous DNA loci were all previously described [Chromosome 7, DNA segment, Washington 12 (D7Was12); the parathyroid hormone (Pth); calcitonin (Calc); hemoglobin, β chain (Hbb); metallothionein-I (Mt-1); adenine phosphoribosyltransferase (Aprt); growth hormone receptor (Ghr); prostaglandin E receptor EP2 subtype (Ptgerep2); dihydrofolate reductase-2 (Dhfr2); fibroblast growth factor a (Fgfa); and glucocorticoid receptor-1 (Grl-1)].

Comparison of the haplotype distribution of protocadherin genes with those determined for loci throughout the mouse genome allowed each to be mapped to specific regions of mouse chromosomes. The probability for linkage was >99% and indicated assignment of both the pc42 gene and the pc43 gene was chromosome 18. The assignment of the protocadherin 413 gene was chromosome 7. The region of chromosome 18 to which the pc42 and pc43 genes were mapped corresponds to the ataxia (ax) loci [Burt, Anat. Rec., 196: 61-69 (1980) and Lyon, J. Hered., 46: 77-80 (1955)] and twirler (Tw) loci [Lyon, J. Embryol. Exp. Morphol., 6: 105-116 (1958)], while the region of chromosome 7 to which the protocadherin 413 gene was mapped corresponds to the shaker (sh-1) locus [Kikuchi et al., Acta Oto-Laryngol., 60: 287-303 (1965) and Lord et al., Am. Nat., 63: 453-442 (1929)]. These loci have been implicated as involved in hereditary neural disease in the mouse. This result is consistent with in situ hybridization results (see Example 12) showing that pc42 and pc43 are strongly expressed in the brain and particularly in the cerebellum.

### EXAMPLE 4

Two additional novel human protocadherin cDNAs and one additional novel rat protocadherin cDNA were isolated using rat protocadherin fragments described in Example 1 as probes.

Initially, the rat clone RAT-214 (SEQ ID NO: 7) was used as a probe to screen a rat brain cDNA library (Stratagene, La Jolla, Calif.). The final washing step was performed twice at 50° C. in 0.1×SSC with 0.1% SDS for 15 minutes. Various clones were identified which contained partial cDNA inserts encoding related protocadherin amino acid sequences. The nucleotide sequence of one novel rat clone designated #6-2 is set out in SEQ ID NO: 108. The first fifteen nucleotides of SEQ ID NO: 108 are the sequence of a linker and are not part of the rat #6-2 clone.

A human fetal brain cDNA library obtained from Stratagene was screened with the 0.7 kbp PstI fragment of clone #6-2. The fragment appears to encode the EC-2 and EC-3 of the rat protocadherin. After screening about 2×10⁶ phages, eleven positive clones were isolated. Sequencing of the clones identified a novel full length human protocadherin cDNA designated human pc3. The nucleotide and deduced amino acid sequence of human pc3 are set out in SEQ ID NOs: 109 and 110.

The 0.7 kbp PstI fragment of rat clone #6-2 was also used to rescreen the Stratagene rat brain cDNA library for full length rat cDNA clones. A clone containing an insert encoding a full length novel protocadherin cDNA was isolated. The DNA and deduced amino acid sequence of the insert are set out in SEQ ID NO: 111 and 112. The fill length rat cDNA was named pc5 because it does not appear to be the homolog of the human pc3 clone based upon a comparison of the sequences.

Concurrently, the 0.8 kbp Eco RI-Pst I fragment of partial rat cDNA designated #43 (SEQ ID NO: 113), which was obtained by screening the Stratagene rat brain cDNA library with a probe corresponding to the human pc43 cytoplasmic domain, was used to probe the Stratagene human cDNA library for full length human protocadherin cDNAs. The fragment appears to encode EC-3 through the beginning of EC-6 of clone #43. One partial clone identified encodes a novel human protocadherin named human pc4. The nucleotide sequence and deduced amino acid sequences of the human pc4 clone are set out in SEQ ID NOs: 114 and 115. The amino acid sequence encoded by the pc4 clone appears to begin in the middle of EC-2 of pc4 and continues through the cytoplasmic tail of the protocadherin.

### EXAMPLE 5

The full length human cDNAs encoding pc42 and pc43 were expressed in L cells (ATCC CCL 1) using the pRC/RSV expression vector (Invitrogen, San Diego, Calif.). The cDNAs were isolated from the Bluescript SK(+) clones described in Example 2 by digestion with SspI followed by blunt-ending with DNA polymerase and digestion with XbaI (for pc42), or by double digestion with SpeI and EcoRV (for pc43). The pRC/RSV expression vector was digested with HindIII, followed by blunt-ending and re-digestion with XbaI for insertion of pc42 sequences, or by digested with XbaI followed by blunt-ending and re-digestion with SpeI for insertion of pc43 sequences. The isolated protocadherin DNAs were ligated into the linearized pRC/RSV vector. The resulting pc42 expression plasmid designated pRC/RSV-pc42 (ATCC 69162) and pc43 expression plasmid designated pRC/RSV-pc43 (ATCC 69163) were purified by CsCl gradient centrifugation and transfected into L cells by a Ca-phosphate method.

The pc42 and pc43 transfectants were morphologically similar to the parental cells. Northern blot analysis of L cells transfected with pc42 or pc43 DNA sequences showed that the transfected cells expressed mRNAs of a size expected to encode the particular protocadherin.

### EXAMPLE 6

Rabbit polyclonal antibodies specific for pc42 and pc43 were generated as well as a mouse monoclonal antibody specific for pc43.

Preparation of Polyclonal Antibodies Specific for pc42 and pc43

DNA sequences encoding portions of the extracellular domain of pc42 and pc43 were each fused to a maltose binding protein-encoding sequence and expressed in bacteria. Specifically, DNAs corresponding to EC-4 through EC-7 of pc42 and EC-3 through EC-5 of pc43 were prepared by PCR and subcloned in the correct reading frame into the multicloning site of the pMAL expression vector (New England Biolabs, Beverly, Mass.) which contains sequences encoding maltose binding protein immediately upstream of the multicloning site. The resulting plasmids were then introduced into E. coli NM522 cells (Invitrogen, San Diego, Calif.) by a single step transformation method. Expression of the fusion proteins was induced by the addition of IPTG and the fusion proteins were purified from cell extracts by amylose resin affinity chromatography (New England Biolabs) as described by the manufacturer. The fusion proteins were used for the immunization of rabbits without further purification.

Polyclonal antibodies were prepared in rabbits by immunization at four subcutaneous sites with 500 μg of purified fusion protein in Freund's complete adjuvant. Subsequent immunizations with 100 μg of the fusion protein were in Freund's incomplete adjuvant. Immune sera was passed through sepharose coupled to maltose binding protein (New England Biolabs) and polyclonal antibodies were purified from immune sera using Sepharose affinity columns prepared by reaction of the purified fusion protein with CNBr Sepharose (Pharmacia). Reactivity of the polyclonal sera with purified pc42 fusion protein and pc42 transfected cell extracts (described in Example 5) was confirmed.

Preparation of Monoclonal Antibodies Specific for pc43

The pc43 fusion protein (containing the EC-3 through EC-5 subdomains of pc43) was used to generate monoclonal antibodies in mice according to the method of Kennett, Methods in Enzymol., 58:345-359 (1978). Briefly, mice were immunized with the pc43 fusion protein (100 μg) at two subcutaneous sites. The spleen from the highest titer mouse was fused to the NS1 myeloma cell line. The resulting hybridoma supernatants were screened in a ELISA assay for reactivity with the pc43 fusion protein and with maltose binding protein. The fusion wells with the highest reactivity to the pc43 extracellular domains were subcloned. The hybridoma cell line designated 38I2C (ATCC HB 11207) produced a IgG₁ subtype monoclonal antibody specific for pc43. Reactivity of the monoclonal antibody produced by hybridoma cell line 38I2C to pc43 was confirmed by immunoblotting the pc43 L cell transfectants described in Example 5. The 38I2C monoclonal antibody is specific for human pc43.

### EXAMPLE 7

L cells transfected with DNA sequences encoding pc42 and pc43 as prepared in Example 5 were assayed for expression of the protocadherins by immunoblot and by immunofluorescence microscopy.

Immunoblot Analysis

Cell extracts of pc42 and pc43 transfectants were subjected to SDS-PAGE and then blotted electrophoretically onto a PVDF membrane (Millipore, Bedford, Mass.). The membranes were incubated with 5% skim milk in Tris-buffered saline (TBS) for two hours and then respectively with either pc42 polyclonal sera or pc43 monoclonal antibody for one hour. The membranes were washed three times (for 5 minutes each wash) with TBS containing 0.05% Tween 20 and repectively incubated with alkaline phosphatase-conjugated anti-rabbit IgG antibody or anti-mouse IgG antibody (Promega, Madison, Wis.) in the same buffer for one hour. After washing the membranes with TBS containing 0.05% Tween 20, reactive bands were visualized by using Western Blue solution (Promega).

Anti-pc42 polyclonal antibodies stained a band of about 170 kDa molecular weight in pc42 transfected cells, but not parental L cells. The pc43-specific monoclonal antibody (38I2C) and polyclonal antibodies stained two adjacent bands of about 150 kDa molecular weight in pc43 transfected cells. The pc43 antibodies did not stain bands in parental L-cells. The molecular weights indicated by the staining of bands by the pc42 and pc43 antibodies are significantly larger than the molecular weights predicted from the deduced amino acid sequences. This discrepancy in molecular weight is common among various cadherin-related proteins and may be attributable to the glycosylation and/or cadherin specific structural properties. The pc42 antibody also stained smaller bands, which may be proteolytic degradation products.

When transfected cells were trypsinized and cell extracts were prepared, run on SDS/PAGE and immunoblotted with the appropriate antibody, the pc42 and pc43 polypeptides expressed by the transfected cells were found to be highly sensitive to proteolysis and were easily digested by 0.01% trypsin treatment. In contrast to the classic cadherins, however, these proteins were not protected from the digestion in the presence of 1-5 mM Ca²⁺.

Immunofluorescence Microscopy

Transfected cells were grown on a cover slip precoated with fibronectin and were fixed with 4% paraformaldehyde for 5 minutes at room temperature or with cold methanol on ice for 10 minutes followed by 4% paraformaldehyde fixation. After washing with TBS, the cells were incubated with TBS containing 1% BSA for 30 minutes and then with anti-pc42 polyclonal antibody or anti-pc43 monoclonal antibody in TBS containing 1% BSA for 1 hour at room temperature. Cover slips were then washed with TBS containing 0.01% BSA and respectively incubated with FITC-conjugated anti-rabbit antibody or anti-mouse antibody (Cappel, Durham, N.C.) for 60 minutes at room temperature. The cells were washed again with TBS containing 0.01% BSA and subjected to fluorescence microscopy. Both pc42-specific and pc43-specific polyclonal antibodies stained the cell periphery of transfected cells expressing the protocadherin proteins, mainly at the cell-cell contact sites. The antibodies did not stain the parent L cells, nor did rabbit preimmune sera stain the pc42 and pc43 transfectants.

### EXAMPLE 8

The cell aggregation properties of the transfected L cells expressing protocadherin proteins were examined. Transfected L cells were cultured in Dulbecco's Modified Eagles Medium (DMEM) (Gibco, Grand Island, N.Y.) supplemented with 10% fetal bovine serum at 37° C. in 5% CO₂. Cells grown near confluence were treated with 0.01% trypsin in the presence of 1 mM EGTA for 25 minutes on a rotary shaker at 37° C. and collected by centrifugation. The cells were washed three times with Ca²⁺ free HEPES-buffered saline (HBS) after adding soybean trypsin inhibitor, and were resuspended in HBS containing 1% BSA. The cell aggregation assay [Urushihara et al., Dev. Biol., 70: 206-216 (1979)] was performed by incubating the resuspended cells in a 1:1 mixture of DMEM and HBS containing 1% BSA, 2 mM CaCl₂ and 20 μg/ml of deoxyribonucelease on a rotary shaker at 37° C. for 30 minutes to 6 hours.

The pc42 and pc43 transfectants did not show any significant cell aggregation activity during periods of incubation less than 1 hour. This is in contrast to the cell aggregation that occurs with classic cadherins in similar experiments (Nagafuchi et al., supra, and Hatta et al., supra). However, prolonged incubation of transfected cells (more than 1-2 hours) resulted in gradual re-aggregation of the cells into small aggregates. Similar results were obtained when single cell suspensions of transfected cells were prepared by trypsin treatment in the presence of Ca²⁺. No re-aggregation was observed under the same conditions when untransfected L cells or L cells transfected with pRC/RSV vector alone were tested. When pc43 transfectants labelled with DiO (Molecular Probes, Eugene, Oreg.) were incubated with unlabelled pc42 transfectants in the cell aggregation assay, aggregation of labelled and unlabelled cells was almost mutually exclusive indicating that protocadherin binding is homophilic.

In view of the fact that the protocadherin cytoplasmic domains exhibit no apparent homology to cadherin domains, experiments were performed to determine if the difference in cytoplasmic domains could account for the difference in cell aggregation activity observed in cadherin and protocadherin transfectants. The cytoplasmic domain of pc43 was replaced with the cytoplasmic domain of E-cadherin and aggregation of cells transfected with the chimeric construct was analyzed.

The Bluescript SK(+) clone described in Example 2 which contained the entire coding sequence for pc43 was digested with EcoRV and then partially digested with XbaI to remove the sequence corresponding to the cytoplasmic domain, and the plasmid DNA was purified by agarose gel electrophoresis. The cDNA corresponding to the cytoplasmic domain of mouse E-cadherin was synthesized by PCR using mouse cDNA made from mouse lung mRNA as a template and specific primers corresponding to a region near the N-terminus of the cytoplasmic domain sequence or the region containing the stop codon of mouse E-cadherin (Nagafuchi et al., supra). A XbaI sequence was included to the 5′ end of the upstream primer. The E-cadherin cytoplasmic domain cDNA was then subcloned into the linearized pc43 Bluescript clone. The DNA containing the entire resulting chimeric sequence was cut out with SpeI and EcoRV and was subcloned into the SpeI-blunted XbaI site of the expression vector pRc/RSV vector. Finally, L cells were transfected with the resultant construct by a calcium phosphate method. After screening with G418 for about 10 days, the transfectants were stained with FITC-labeled 38I2C anti-pc43 antibody and subjected to FACS analysis. A portion of highly labeled cells were isolated and cloned. Transfectants showed a morphology similar to that of parental L cells and the expressed protein was localized at the cell periphery using pc43 antibody for immunofluorescence microscopy.

Cell aggregation activity of the chimeric transfectants was analyzed as follows. The chimeric pc43 transfectants were labeled with DiO for 20 minutes at room temperature. The resultant cells were trypsinized in the presence of 1 mM EGTA and single cell suspension was made. Then, the cells were mixed with unlabeled other type of transfectants and incubated on a rotary shaker for two hours. The results were examined with a fluorescence and a phase contrast microscope apparatus. Antibody inhibition of cell aggregation was examined by incubation of the transfectants in the presence of polyclonal anti-pc43 antibody (100 ng/ml) in the standard assay medium.

In the cell aggregation assay, the chimeric pc43 transfectants showed clear Ca²⁺-dependent cell aggregation within forty minutes of incubation. Cell aggregation was inhibited by the addition of pc43-specific polyclonal antibody.

### EXAMPLE 9

The procedures of Maruyama et al., J. Biochem., 95: 511-519 (1984) were used to determine the calcium binding properties of pc43 by Western blot analysis in the presence or absence of calcium-45. The pc43 fusion protein described in Example 6 containing pc43 subdomains EC-3 through EC-5 was compared to the calcium binding protein calmodulin. Samples of purified pc43 fusion protein were run on SDS/PAGE and electrophoretically transferred to PVDF membrane. Binding of the ⁴⁵Ca²⁺ to the pc43 fusion protein was detected by autoradiography and was determined to be nearly as efficient as binding of⁴⁵Ca²⁺ to calmodulin. In contrast, there was no binding of calcium to purified maltose binding protein lacking the pc43 extracellular domain. The pc43 subdomains EC-3 through EC-5 contain sequences highly homologous to the putative Ca^{2±} binding motifs found in E-cadherin. [See, Ringwald et al., EMBO J., 6: 3647-3653 (1987).]

### EXAMPLE 10

The expression of mRNA encoding pc42 and pc43 was assayed in various tissues and cell lines by Northern blot.

Total RNAs were prepared by the guanidium isothiocyanate method and poly(A) + RNAs were isolated using a FastTrack kit (Invitrogen). RNA preparations were electrophoresed in a 0.8% agarose gel under denaturing conditions and transferred onto a nitrocellulose filter using a capillary method. Northern blot analyses were performed according to the method of Thomas, Proc. Natl. Acad. Sci. USA, 77: 5201-5205 (1980). The final wash was in 0.2× standard saline citrate containing 0.1% sodium dodecyl sulfate at 65° C. for 10 minutes.

Protocadherin mRNA Expression in Adult Rat Tissues

Total mRNA preparations of rat tissues including brain, heart, liver, lung, skin, kidney and muscle were separated electrophoretically under denaturing conditions (10 μg mRNA/lane) and transferred onto nitrocellulose filters. The filters were hybridized with ³²P-labelled cDNA fragments MOUSE-326 (which corresponds to EC-4 of human pc42) and RAT-218 (which corresponds to EC-5 of human pc43). The mRNAs of both protocadherins were highly expressed in brain. The pc42 probe detected a major band of 7 kb and a minor band of 4 kb in size, possibly representing the products of alternative splicing. The pc43 probe hybridized to a major band of 5 kb in size and with minor bands of smaller sizes.

Developmental Expression of Protocadherin mRNA in Rat Brain

To examine the developmental regulation of mRNA expression of days 5 and 11 and from adult rats was prepared and subjected to Northern blot analysis as described above for other rat tissues. β-actin was used as an internal standard. mRNA levels for pc42 and pc43 proteins increased during embryonic development of the brain as compared with β-actin expression.

Protocadherin mRNA Expression in Human Cell Lines

Several neuronal and glial cell lines (including human SK-N-SH neuroblastoma, human U251 glioma, and mouse Neuro-2a neuroblastoma cell lines) were assayed by Northern blot using ³²P-labelled for expression of pc42 and pc43 mRNA. Human cell lines were probed with HUMAN-42 (which corresponds to EC-4 of human pc42) and HUMAN-43 (which corresponds to EC-5 of human pc43) cDNA fragments while the mouse cell line was probed with MOUSE-326 (which corresponds to EC-4 of human pc42) and RAT-322 (which corresponds to EC-5 of human pc43) cDNA fragments. SK-N-SH human neuroblastoma cells and U251 human glioma cells were found to express pc43 mRNA and Neuro-2a mouse neuroblastoma cells were found to express pc42 mRNA.

### EXAMPLE 11

Expression of pc43 protein in various tissues, extracts and cells was assayed by Western blot and immunofluorescence microscopy.

Expression in Rat Cardiac Muscle Extracts

A rat heart non-ionic detergent extract was prepared by freezing a heart in liquid nitrogen after removal, powdering in a mortar and pestle, grinding briefly in a polytron in 0.5% Nonidet P40 in [10 mM PIPES (pH 6.8), 50 mM NaCl, 250 mM NH₄SO₄, 300 mM sucrose, 3 mM MgCl₂] and microfuging for 15 minutes. Samples were separated by SDS/PAGE and electrophoretically transferred to nitrocellulose (Towbin et al., PNAS 76:4350-4354, 1979). Two pc43 protein bands with molecular weights of 150 KDa and 140 KDa were detected with rabbit polyclonal antibodies to pc43 by the immunoblot method described in Example 7.

Expression in Tissue Sections and Cells

To determine the localization of the protocadherins in various tissues, human and rat adult tissues were removed, incubated in 30% sucrose in PBS for 30 minutes at 4° C., embedded in OCT compound (Tissue-Tek, Elkhart, Ind.) in cryomolds and quickly frozen. Six micron sections were cut and placed on glass slides. The slides were washed with PBS and fixed in 3% p-formaldehyde for 5 minutes. To permeablize the tissue sections, the slides were immersed in −20° C. acetone for 10 minutes and air dried. The sections were blocked with 2% goat serum and 1% BSA in PBS for 30 minutes and then incubated with the rabbit anti-pc43 polyclonal antisera for 1 hour at room temperature. The sections were rinsed 3 times in PBS containing 0.1% BSA and incubated with a biotinylated anti-rabbit (Vector Laboratories, Burlingame, Calif.) in 1% BSA in PBS for 30 minutes. After rinsing 3 times, strepavidin-conjugated with FITC (Vector Laboratories) was added for 30 minutes and again washed 3 times. For co-localization studies, an appropriate primary antibody was used with a TRITC-conjugated secondary antibody.

A. Muscle

Immunolocalization of pc43 in rat cardiac muscle shows that pc43 is localized in a repeating pattern which is consistent with pc43 being associated with the sarcomeres. Sarcomeres are repetitive contractile units between the fascia adherens in skeletal and cardiac muscle. Co-localization with cytoskeletal proteins shows that pc43 is present at the ends of the sarcomeres in the Z lines which are associated with desmin and the actin-binding protein vinculin, and alpha-actinin. The thin microfilaments of F-actin are associated with the thick myosin filaments between the Z lines. In contrast, N-cadherin is localized at the ends of cardiac myocytes at the fascia adherens junctions at sites of mycocyte:myocyte contact. The localization of pc43 in cardiac muscle suggests that pc43 may play a role in muscle contraction in the anchoring of the contractile apparatus to the plasma membrane.

Similar localization for pc43 was observed in rat skeletal muscle. Ultrastructural studies have shown that dystrophin, the gene product lacking in Duchenne muscular dystrophy, is a component of the sarcolemma Porter et al., J. Cell. Biol., 117:997-1005 (1992)]. The sarcolemma is connected to the contractile apparatus at the M and Z lines where pc43 is localized.

B. Brain

Reactivity of anti-pc43 polyclonal antibody and monoclonal antibody 38I2C on frozen sections of rat and human cerebellum, respectively, shows that the major sites of pc43 expression are located in Purkinje cells and the granule cell layer which contains numerous small neurons.

C. Placenta

Strong reactivity of monoclonal antibody 38I2C with human syncytiotrophoblasts was also observed in development of the placenta at an early state (5-7 weeks of gestation). Expression appeared to gradually decrease as the stage progressed indicating that pc43 may be involved in the implantation of fertilized eggs into the placenta.

D. Neuroblastoma and Astrocytoma Cells

Immunocytochemical localization of pc43 in Sk-N-SH neuroblastoma cells and UW28 astrocytoma cells using anti-pc43 antibodies reveals a punctate cell surface distribution of pc43 and in some cells there is a localization at the tips of extensions of neuronal foot processes. At sites of cell-cell contact of UW28 astrocytoma cells, pc43 is organized in a series of parallel lines. The lines start at the contact site and extend approximately 5 micron. F-actin microfilaments were identified with rhodamine-phalloidin (Molecular Probes, Eugene, Oreg., as described by the manufacturer) showing that the microfilaments in the cell appear to end in the pc43 linear structures which extend from the edge of the cell at sites of cell contact.

Immunoblotting studies with pc43 specific antibodies show that a protein with a molecular weight of 140 kDa is recognized in human Sk-N-SH neuroblastoma cells and in UW28 astrocytoma cells.

E. Osteoblasts

Immunocytochemical localization of pc43 using monoclonal antibody 38I2C in tow human ostogenic sarcoma cell lines [SaOS (ATCC HTB 85) and MG-63 (ATCC CRL 1427)] and in cultures of normal human trabecular osteoblasts [culture system described in Civitelli et al., J. Clin. Invest., 91: 1888-1896 (1993)] showed that pc43 is expressed in osteoblasts in a pattern similar to that seen in UW28 astrocytoma cells. At sites of cell-cell contact, pc43 is organized in a series of parallel lines that appear to correspond to the actin stress fibers. In addition, in some cells, pc43 appears to localize at the tips of contacting cell processes. Northern blot analysis provides additional evidence that pc43 is expressed in normal human trabecular osteoblasts. A pc43 specific DNA probe hybridized to a major band of 5 kb in samples of poly-A mRNA isolated from normal human trabecular osteoblasts.

### EXAMPLE 12

In situ hybridization experiments using protocadherin specific RNA probes were performed on cryosections of rat tissue.

Sense and antisense ³⁵S-riboprobes were made using the standard procedure described by Promega (Madison, Wis.). An approximately 400 bp EcoRI-XbaI fragment of the MOUSE-326 cDNA clone was used as a pc42 specific probe. This fragment encodes the middle of EC-3 to the end of EC-4 of pc42. An approximately 700 bp SmaI fragment of the RAT-218 cDNA clone was used as a pc43 specific probe. The fragment encodes the end of EC-3 to the end of EC-5 of pc43.

Rat adult tissues were harvested and immediately embedded with OCT Compound (Tissue-Tek) in cryomolds and quickly frozen in a bath of 95% ethanol/dry ice The frozen [symbol] tissue sections were cut using a cryostat (Reichert-Jung, Model #2800 Frigocut N, Leica, Inc., Gilroy, Calif.). Cut tissue sections were stored at −80° C.

The in situ protocol used was a variation of that described by Angerer et al., Methods in Enzymology, 152: 649-660, (1987). All solutions were treated with diethylpyrocarbonate (DEPC, Sigma, St. Louis, Mo.) to remove RNase contamination. The tissue sections were first fixed in 4% paraformaldehyde at 4° C. for 20 minutes. To remove excess paraformaldehyde and stop the tissue fixation, the slides were washed in PBS (phosphate buffered saline), denatured in a graded series of alcohols (70, 95, 100%) and then dried. To prevent the tissue from detaching from the glass slide during the in situ procedure, the tissue sections were treated in a poly-L-lysine solution (Sigma) at room temperature for 10 minutes. To denature all RNA in the tissue, the sections were placed in a solution of 70% formamide/2×SSC (0.15 M NaCl/0.3 M Na citrate, pH 7.0) at 70° C. for 2 minutes after which they were rinsed in chilled 2×SSC, dehydrated in a graded series of alcohols and then dried. Once dried, the sections were prehybridized in hybridization buffer [50% formamide/50 mM DTT (dithiothrietol)/0.3M NaCl/20 mM Tris, pH 8.0/5 mM EDTA/1× Denhardt's (0.02% Ficoll Type 400/0.02% polyvinylpyrrolidone/0.02% BSA)/10% Dextran Sulfate] at the final hybridization temperature for approximately 4 hours. After prehybridization, approximately 1×10⁶ cpm of the appropriate riboprobe was added to each section. The sections were generally hybridized at 45° C. overnight (12-16 hours). To insure that the hybridization seen was specific, in some experiments the hybridization stringency was increased by raising the hybridization temperature to 50° C. As both the 45° C. and 50° C. experiments gave comparable results, the standard hybridization temperature used was 45° C.

To remove excess, nonhybridized probe, the sections were put through a series of washes. The sections were first rinsed in 4×SSC to remove the bulk of the hybridization solution and probe. Next a 15 minute wash in 4×SSC/50 mM DTT was carried out at room temperature. Washes at increased stringencies were also utilized. A 40 minute wash in 50% formamide/2×SSC/50 mM DTT was performed at 60° C. Four final room temperature washes were carried out for 10 minutes each: two in 2×SSC and two in 0.1×SSC. The washed slides were dehydrated in a graded series of alcohols and dried.

To visualize the hybridized probe, the slides were dipped in Kodak NTB2 nuclear emulsion (International Biotechnology, New Haven, Conn.) which had been diluted 1:1 in dH₂O. Once dry, the slides were stored at 4° C. in light-tight boxes for the appropriate exposure time. The in situ slides were independently viewed by two persons and scored positive or negative for hybridization signal.

All in situ hybridization studies were performed on rat tissue. Because results from Northern blot experiments (see Example 9) indicated that both pc42 and pc43 are expressed in adult brain, in situ hybridization studies were carried out to localize the expression of these molecules to specific brain cell types. Hybridization seen in the normal adult rat brian was specific (no background hybridization was seen with the sense probes) and was localized to specific regions in the brain. The overall pattern of expression seen for pc42 and pc43 was very similar, with the major difference being in the level of expression. pc43 appears to be expressed at a lower level than pc42. Both molecules are expressed in the germinal and pyramidal cells of the hippocampus, Purkinje cells of the cerebellum and neurons in grey matter. In addition, pc42 is expressed in glial cells in the white matter but, in contrast to the expression of pc43 in glioma cell lines (as described in Example 9), expression of pc43 in normal glial cells was not observed. In the spinal chord, both protocadherins are expressed in the motor neurons in the gray matter and pc42 is expressed in the glial cells in the white matter.

When expression of both protocadherin molecules was analyzed in brains and spinal chords from rats having EAE (experimental allergic encephalomyelitis) [Vandenbark et al., Cell. Immunol., 12: 85-93 (1974)], the same structures as described above [symbol] expression of pc42 was observed in the leukocytic infiltrates in the EAE tissues. Expression of pc42 in leukocytes was confirmed by in situ hybridization analysis of two leukocytic cell lines, RBL-1 and y3.

Expression of both protocadherin-42 and -43 was observed in the developing brain of rat embryos at all embryological days tested (E15-E19). In addition protocadherin-43 was observed in the developing rat heart at all embryological days tested (E13-E19). This finding is consistent with the immunohistochemistry results showing protocadherin-43 expression in adult heart.

To determine possible roles of protocadherins in the development of the nervous system, expression profiles of protocadherin members in developing rat brain and adult rat brain were also examined by in situ hybridization. A series of coronal, sagittal and horizontal sections of rat brains at postnatal days 0, 6, 14, 30 (P0 through P30) and at 3 months (young adult) were hybridized with labelled cRNA probes corresponding to various protocadherins of the invention including pc42, pc43, RAT-212, RAT-411, and RAT-418. In developing brain, RAT-411 was expressed at high levels in neurons of the olfactory bulb, i.e., mitral cells and periglomerular cells. The expression of RAT-411 mRNA was transient; expression appeared at P0, peaked at P6, diminished by P14, and was undetectable at P30 and in adult brain. In the adult, pc43 mRNA was found to be expressed predominantly in Purkinje cells in the cerebellum. The expression of pc43 mRNA in Purkinje cells was observed from the beginning of Purkinje cell differentiation at around P6. Other protocadherin members were expressed at very low levels in various areas of developing and adult brains. These results indicate that protocadherin members are differentially expressed during the development of the central nervous system, and suggest that RAT-411 and pc43 have specific roles during the development of olfactory bulb neurons and Purkinje cells, respectively.

### EXAMPLE 13

Conventional immunoprecipitations using pc43-specific polyclonal antibodies and monoclonal antibody 38I2C were performed to identify proteins that interacted with pc43 in L cell transfectants.

The pc43 and chimeric pc43 transfectants were metabolically labeled by incubating the cells in Dulbecco's modified Eagle's medium containing [³⁵S] methionine (50 uCi/ml) overnight. After washing, the transfectants were lysed with PBS containing Triton X 100 and incubated with anti-pc43 antibody. The immunocomplexes were then collected using protein A-Sepharose beads. The resulting beads were washed five times with a washing buffer (50 mM Tris-HCl, pH 8.0, containing 0.5M NaCl, 0.1% ovalbumin, 0.5% NP-40, 0.5% Triton X 100 and 1 mM EDTA) at room temperature. Protein was separated by SDS-PAGE and subjected to autoradiography.

The chimeric pc43 co-precipitated with 105 kDa and a 95 kDa bands that are likely to correspond to α- and β-catenins, respectively, because anti-α-catenin and anti-β-catenin antibodies stained comparable bands. Pc43, on the other hand, co-precipitated with a 120 kDa band.

While the present invention has been described in terms of specific methods and compositions, it is understood that variations and modifications will occur to those skilled in the art. Therefore, only such limitations as appear in the claims should be placed on the invention.

1. A purified and isolated polynucleotide sequence encoding human protocadherin pc3.

2-3. (canceled)

4. The polynucleotide sequence of claim 1 which is a DNA sequence.

5. The DNA sequence of claim 4 which is a cDNA sequence.

6. The DNA sequence of claim 4 which is a genomic DNA sequence.

7. The DNA sequence of claim 4 which is wholly or partially chemically synthesized.

8. A polynucleotide sequence according to claim 1 comprising the human protocadherin pc3 encoding sequence of SEQ ID NO: 109.

9-10. (canceled)

11. A biologically functional DNA vector comprising a DNA sequence according to claim 4.

12. The vector of claim 11 wherein said DNA sequence is operatively linked to an expression control DNA sequence.

13. A host cell transformed or transfected with a DNA sequence according to claim 4 in a manner allowing the expression in said host cell of a protocadherin polypeptide.

14. A method for producing a protocadherin polypeptide comprising the steps of growing a host cell according to claim 13 in a suitable nutrient medium and isolating protocadherin polypeptide from said cell or from the medium of its growth.

15. Purified and isolated human protocadherin pc3 polypeptide.

16-17. (canceled)

18. An antibody substance specific for human protocadherin pc3.

19-20. (canceled)

21. The antibody substance of claim 18 which is a monoclonal antibody.

22. A hybridoma cell line producing a monoclonal antibody according to claim 21.

23. A method for modulating the binding activity of human protocadherin pc3 comprising contacting said protocadherin with an antibody substance according to claim 18 specific for said protocadherin.

24-28. (canceled)
